(19) United States
(12) Patent Application Publication
(10) Pub. No.: US 2023/0083808 A1
(43) Pub. Date: Mar. 16, 2023

(54) SUTURE NEEDLES WITH FLEXIBLE AND NON-FLEXIBLE COMPONENTS

(71) Applicant: Ergosurgical Group Corp., Santa Maria, PR (US)

(72) Inventors: Luis Jose Almodovar, Santa Maria, PR (US); Mark A. Follman, Glen Rock, NJ (US)

(21) Appl. No.: 17/945,812

(22) Filed: Sep. 15, 2022

Related U.S. Application Data
(60) Provisional application No. 63/244,556, filed on Sep. 15, 2021.

(51) Int. Cl. A61B 17/06

(52) U.S. Cl. CPC A61B 17/06066; A61B 17/06166; A61B 2017/06176; A61B 2017/06028; A61B 2017/061; A61B 2017/0608

(57) ABSTRACT

Disclosed is a suturing needle having a thread attachment portion and a ramp portion comprising a tapered portion extending from a proximal end of the needle to cover at least a portion of a suture thread swaged in the thread attachment portion. The tapered portion of the ramp portion provides a smooth transition from a diameter of the suture thread to a diameter of the needle body. The tapered transition portion allows for engaging the suture needle on rollers of a rotational needle driver in one smooth motion. The suturing needle includes a hollow flexible extension component having a distal end and a proximal end, the distal end connected to the proximal end of the thread attachment component, and the proximal end attached to the distal end of the tapered extension ramp portion. The suturing needle can include a combination of flexible and non-flexible sections and/or a combination of straight and curved sections.

### CROSS REFERENCE TO RELATED APPLICATIONS

The present application claims the benefit of U.S. Provisional Patent Application 63/244,556 filed 15 Sep. 2021; which is incorporation herein by reference for all purposes.

### TECHNICAL FIELD

The present disclosure generally relates to suture needles, including but not limited to curved suture needles. The curved suture needles include a flexible extension component, which enables movement in multiple directions, and enables deeper penetration into the ski compared to standard curved suture needles. The present disclosure also include straight suture needles including flexible and non-flexible components.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Suturing procedures used to close wounds or incisions consume a considerable amount of time during a surgical procedure. Generally, the instruments used in suturing procedures are the suturing filament material, the suturing needle, and the suturing needle driver. Efforts made to reduce the time and enhance the safety of the procedure have mainly focused on modifications to the needle driver.

The traditional way of driving a needle has been to clamp it down between two flat tips of a needle driver. This forms a static relationship between the needle and needle driver because the needle, once clamped down between the flat tips, cannot move relative to the driver. Moreover, needle drivers are generally designed to fit right-handed users. Thus, left-handed users have difficulties performing the suturing procedure. The right handedness of those devices further affects the capacity of left-handed surgeons to lock and unlock the drivers' locking mechanism. This increases the risks of negative outcomes for patients from sub optimal or incorrect needle driver maneuverings.

Recent advances in needle drivers address these deficiencies, including at least this static relationship of the needle and the handedness of the prior art drivers, such as the development of new roller needle drivers.

Current suture needles have not been designed for, and therefore cannot optimally perform, this task because they were not designed to work with new roller needle drivers. When trying to drive standard swaged needles with these new ergonomic rotational action needle drivers, the rollers generally need to be opened to move over the swage end of the needle. That is, the swage represents a step or lip on the needle that may be damaged by the rollers, or conversely, that may damage the rollers.

Accordingly, new needle designs that may enable smooth loading of a swaged needle by these new needle drivers are desirable and an object of the present disclosure.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

### SUMMARY

The present disclosure provides a suturing needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a hollow extension ramp portion having a distal end and a tapered portion at a proximal end, the distal end attached to a proximal end of the thread attachment component; a hollow flexible extension component having a distal end and a proximal end, the distal end connected to the proximal end of the thread attachment component, and the proximal end attached to the distal end of the tapered extension ramp portion; and a suture thread attached to the thread attachment component.

The present disclosure also provides a method of suturing tissues using a roller needle driver, the method comprising the steps of: (a) providing a suture needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a ramp portion having a distal end and a tapered portion at proximal end, the distal end attached to a proximal end of the thread attachment component; a hollow flexible extension component having a distal end and a proximal end, the distal end connected to the proximal end of the thread attachment component, and the proximal end attached to the distal end of the tapered extension ramp portion; and a suture thread attached to the thread attachment component; (b) grasping, between rollers of the roller needle driver, the suture thread or the tapered portion of the ramp portion distal from the needle body; (c) loading the needle so that the rollers are positioned on the needle body without opening the rollers; (d) driving the needle through a tissue being subjected to suture; (e) moving the needle through the tissue without releasing the needle from the rollers; (f) grasping the suture needle at an end exiting from the tissue through which the needle was moved; and (g) repeating steps (d)-(f) until completion of the suturing process.

The system as described herein, both as to its configuration and its mode of operation will be best understood, and additional objects and advantages thereof will become apparent, by the following detailed description of a preferred embodiment taken in conjunction with the accompanying drawing.

It is acknowledged that the term “comprise” may, under varying jurisdictions, be attributed with either an exclusive or an inclusive meaning. For the purpose of this specification, and unless otherwise noted, the term ‘comprise’ shall have an inclusive meaning, i.e., the term “comprise” will be taken to mean an inclusion of not only the listed components it directly references, but also other non-specified components or elements. This rationale will also be used when the term “comprised” or “comprising” is used in relation to one or more steps in a method or process.

When the word “invention” is used in this specification, the word “invention” includes “inventions”, that is, the plural of “invention”. By stating “invention”, the Applicant does not in any way admit that the present application does not include more than one patentable and non-obviously distinct invention and Applicant maintains that the present application may include more than one patentable and non-obviously distinct invention. The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the full scope of the application, which is measured by the claims, nor is it intended to be limiting as to the scope of the disclosure in any way.

### BRIEF DESCRIPTION OF THE DRAWINGS

All of the figures depict preferred embodiments although other embodiments are contemplated, and the present disclosure is not limited to the embodiments shown.

FIG. 1 is an illustration of a curved suture needle according to one embodiment of the present disclosure.

FIGS. 2A-2B are illustrations of a curved suture needle including a flexible extension part according to an exemplary embodiment of the present disclosure.

FIGS. 3A-3C are illustrations of a curved suture needle including a flexible extension part having a laser-cut pattern filled with a polymer according to an exemplary embodiment of the present disclosure.

FIGS. 4A-4C are illustrations of a curved suture needle including a flexible extension part surrounds by a plurality of tubular structures according to an exemplary embodiment of the present disclosure.

FIGS. 5A-5C are illustrations of a curved suture needle including a flexible extension part surrounded by a braided sheath according to an exemplary embodiment of the present disclosure.

FIGS. 6A-6C are illustrations of a curved suture needle including a flexible extension part having a laser-cut pattern according to an exemplary embodiment of the present disclosure.

FIGS. 7A-7B are illustrations of a curved suture needle including a singular flexible extension part according to an exemplary embodiment of the present disclosure.

FIGS. 8A-8C are illustrations of a curved suture needle including a flexible extension part surrounded by polymer or metallic beads according to an exemplary embodiment of the present disclosure.

FIGS. 9A-9C are illustrations of a curved suture needle including a flexible extension part surrounded by beads and tubes according to an exemplary embodiment of the present disclosure.

FIGS. 10A-10C are illustrations of a curved suture needle including a flexible extension part having nanobeads in an inner area thereof according to an exemplary embodiment of the present disclosure.

FIGS. 11A-11C are illustrations of a curved suture needle including a flexible extension part having a polymer tube or sheath according to an exemplary embodiment of the present disclosure.

FIGS. 12A-12C are illustrations of a curved suture needle including an injection-molded flexible extension part according to an exemplary embodiment of the present disclosure.

FIGS. 13A-13C are illustrations of a curved suture needle including a flexible chain link extension part according to an exemplary embodiment of the present disclosure.

FIGS. 14A-14B are illustrations of a curved suture needle including a polymer-filled flexible extension part having a laser-cut tube or sleeve according to an exemplary embodiment of the present disclosure.

FIGS. 15A-15C are illustrations of a curved suture needle including a flexible extension part with a laser-cut tube or sleeve according to an exemplary embodiment of the present disclosure.

FIGS. 16A-16C are illustrations of a curved suture needle including a pellet-filled flexible extension part according to an exemplary embodiment of the present disclosure.

FIGS. 17A-17F are illustrations of a curved suture needle including non-flexible sections according to an exemplary embodiment of the present disclosure.

FIGS. 18A-18B are illustrations of a curved suture needle including non-flexible sections according to an exemplary embodiment of the present disclosure.

FIGS. 19A-19C are illustrations of a curved suture needle according to exemplary embodiments of the present disclosure showing various attachment locations for the suture.

FIGS. 20A-20I are illustrations of a curved suture needle according to exemplary embodiments of the present disclosure showing various attachment types between flexible and non-flexible sections of the needle.

FIGS. 21A-21C are illustrations of a suture needle according to exemplary embodiments of the present disclosure including various straight and curved sections.

FIGS. 22A-22F are illustrations of a suture needle according to exemplary embodiments of the present disclosure including various straight and curved sections.

FIGS. 23A-23F are illustrations of a suture needle according to exemplary embodiments of the present disclosure including various straight and curved sections.

FIGS. 24A-24F are illustrations of a suture needle according to exemplary embodiments of the present disclosure including various straight and curved sections.

FIGS. 25A-25B are illustrations of a suture needle according to exemplary embodiments of the present disclosure including various straight and curved sections.

FIGS. 26A-26P are illustrations of a straight suture needle according to exemplary embodiments of the present disclosure.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

Technical and scientific terms used herein have the meaning commonly understood by one of skill in the art to which the present description pertains, unless otherwise defined. Reference is made herein to various methodologies and materials known to those of skill in the art.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the various parts of the needle or any assembly of them may be used.

Swaged suturing needles typically comprise attachment of a suture thread to an end of a needle body. These needles have been designed for use with standard needle drivers, which clamp the needle between two flat tips and hold the needle in a static position.

A new generation of devices that move the needle through the tissues by the coordinated rotation of two rollers provide a dynamic relationship between the needle and the needle driver, even when the needle is clamped between the rollers. One of the advantages of this dynamic relationship is that the needle can be driven in multiple planes of rotation, such as perpendicular to the long axis of the driver with the tip pointing up or down (standard), parallel with the long axis of the driver with the tip pointing straight back towards the back of the device or straight forward away from the device (non-standard), and any angle in-between. This dynamic relationship is desirable for the surgeon as it enables placement of the needle at non-traditional angles, permitting suturing of difficult anatomical regions or situations, such as restricted, deep and less accessible locations.

According to certain embodiments, a needle comprising a needle tip portion having a needle tip at a distal end for piercing tissue, a straight or curved trunk portion having a distal end connected to a proximal end of the needle tip portion; and a thread attachment portion connected to a proximal end of the trunk portion for connecting a suturing thread, wherein the needle tip portion is formed to become gradually thicker from the needle tip to the proximal end thereof, and wherein the trunk portion is formed to have a substantially circular cross-section having a non-smooth profile. In certain embodiments, the needle may have a smooth profile. In one or more embodiment(s), the needle comprises at least one of a circular, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, cross-section. In one or more embodiment(s), the suturing needle comprises multiple varied cross sections, including any combinations of the foregoing cross-sections.

Some of these suture needles can be used for medical purposes (e.g., suturing). For example, some of these suture needles can be used in transcatheter suturing, transcatheter intracardiac (or another body organ) suturing, and other flexible platform applications (e.g., endoscopic suturing, colonoscopic suturing). For example, rigid shaft embodiments, as commonly used for laparoscopic, video-assisted thoracoscopic surgery (VATS) and robotic surgery can be used to fundamentally transform suture-based tissue approximation/anchoring from a laborious task to a swift semi-automated or fully-automated endeavor. For example, some of these suture needles can be used for an open surgery, a minimally invasive surgery, a laparoscopic surgery, or an end effector robotic surgery. As such, the some of these suture needles can be used for manual surgery or automated surgery. Some examples of surgeries where some of these suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, fetal surgery or bariatric surgery, or among many others in human (e.g., between newborn until 120 years old, male, female) or animal (e.g., mammal, birds, fish, land animals) applications.

Some of these suture needles can be employed in medical or nonmedical settings, whether on an object is animate or inanimate. For example, the object, when animate, can include a tissue, an organ, a body part, whether of human or animal, or others. For example, the tissue can be a muscle tissue, a bone tissue, a nerve tissue, an organ tissue, or others. For example, the object, when inanimate, can include a medical device, a prosthesis, an implantable, a machine, a surgical instrument, or others. For example, some of the non-medical setting can include garment making, fabric stitching, knot applications, sowing, shoe making, or others.

Current swaged suture needles generally include a lip or step-up in the transition from the suture thread to the thread attachment connector, and another lip or step-up in the transition from the thread attachment component to the needle body. Alone or in combination, these transitions pose a problem for the rollers of a rotational needle driver, as the suture thread may be damaged by the rollers, or conversely, the transitions may damage the rollers of the needle driver. For example, the rollers may roll past the thread attachment component of the needle and may clamp down on that region and damage/cut the suture thread.

The present disclosure overcomes these deficiencies by incorporating an extension portion having a tapered portion that provides a smooth transition from the suture thread to the needle body.

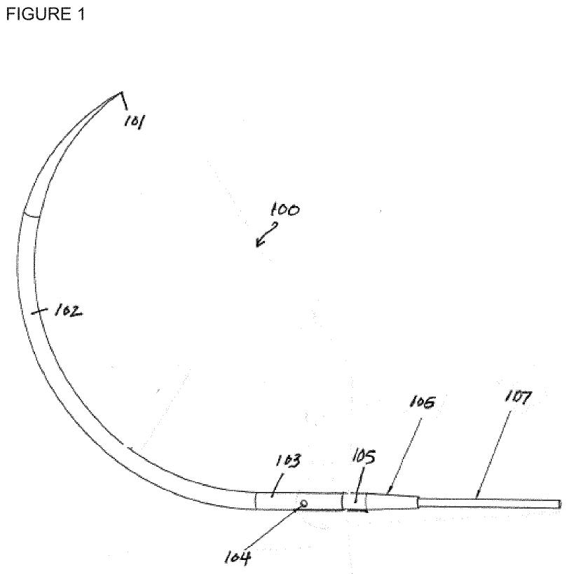

With reference to FIG. 1, a suture needle 100 of the present disclosure may comprise a tip portion 101 at proximal end of suture needle 100 for piercing a tissue, a needle body 102, the proximal end of which extends from the distal end of the needle tip 101, a thread attachment component 103 with a viewing port 104, the proximal end of which extends from the distal end of the needle body 102, an extension portion 105 comprising a tapered portion 106, the proximal end of the extension portion 105 extending from the distal end of the thread attachment component 103, and a suture thread 107. The suture thread 107 is swaged or crimped in the thread attachment component when the suture thread is visible through the viewing port 104. The needle tip portion 101 may be a tapered tip wherein the needle tip portion is round and tapers smoothly to a point, as shown in FIG. 1. Alternatively, the needle tip portion may be triangular, and may have a sharpened cutting edge on the inside or on the outside, or may have a “trocar point” or “taper cut” whereby the needle body is round and tapered, but ends in a small triangular cutting point. Alternatively, the needle tip may be a blunt point, such as for suturing friable tissues, or may have a needle tip portion that includes “side cutting” or “spatula points” whereby the needle is flat on top and bottom with a cutting edge along the front to one side (these are typically used for eye surgery). The needle body 102 is shown to have a curve in the drawings. Exemplary curves include at least half curved or ski, ¼ circle, ⅜ circle, ½ circle, such as shown in the figures, ⅝ circle, and compound curve. Alternatively, the needle body may be straight, such that the present disclosure may comprise a straight needle.

The length of extension portion 105 may be determined by the optimum balance between the minimal length needed by the surgeon to grab the needle by the “extension” when inserting it or withdrawing it to a surgical site, such as through a laparoscopic port, and the maximal length that can feasibly allow tapering to go through tissue following the needle path without causing tissue trauma. In certain embodiments, the tapered portion may comprise a magnetic material or a magnetizable material. In certain embodiments, the suture material may comprise a magnetic material or a magnetizable material.

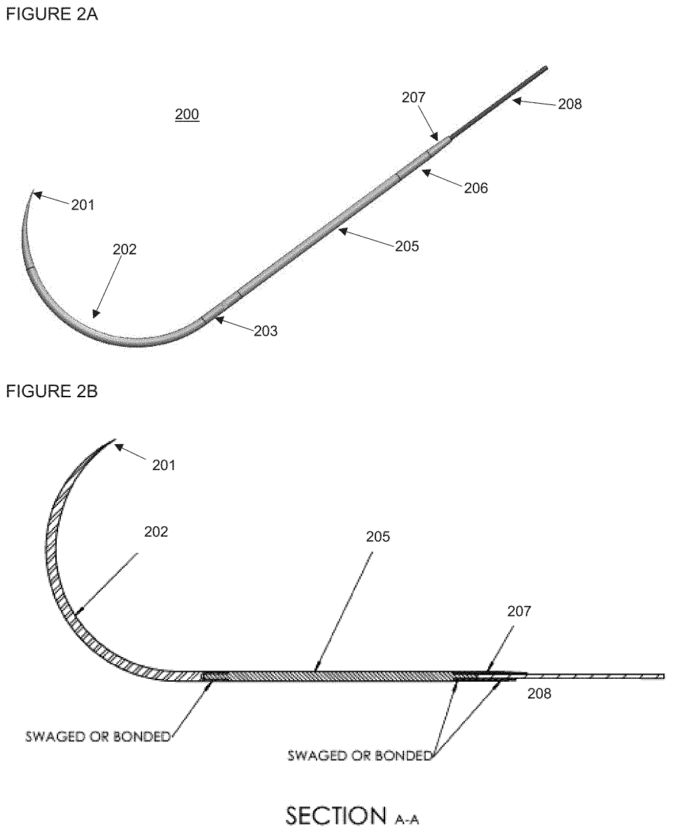

An exemplary embodiment of the curved suture needle 200 is illustrated in FIG. 2A, a side section view thereof is illustrated in FIG. 2B. As illustrated in FIG. 2B, the curved suturing needle 200 comprises a tip portion 201 at a proximal end of suture needle 200 for piercing a tissue, a needle body 202, the proximal end of which extends from the distal end of the needle tip 201, an extension attachment component 203 the proximal end of which extends from the distal end of the needle body 202, a solid one piece flexible extension portion 205, the proximal end of which extends from the distal end of the extension attachment component 203 a ramp portion of the flexible extension portion 206 comprising a tapered portion 207, the proximal end of the ramp portion 206 extending from the distal end of the solid one piece flexible extension portion 205, and a suture thread 208. In the embodiment illustrated in FIG. 2B, the materials of the solid one piece flexible extension portion 205 may be a nickel-titanium alloy or a flexible polymer. The flexible solid one piece extension portion 205 is attached to swaged, bonded or crimped at either end to the extension attachment component 203 and the suture thread 208, respectively.

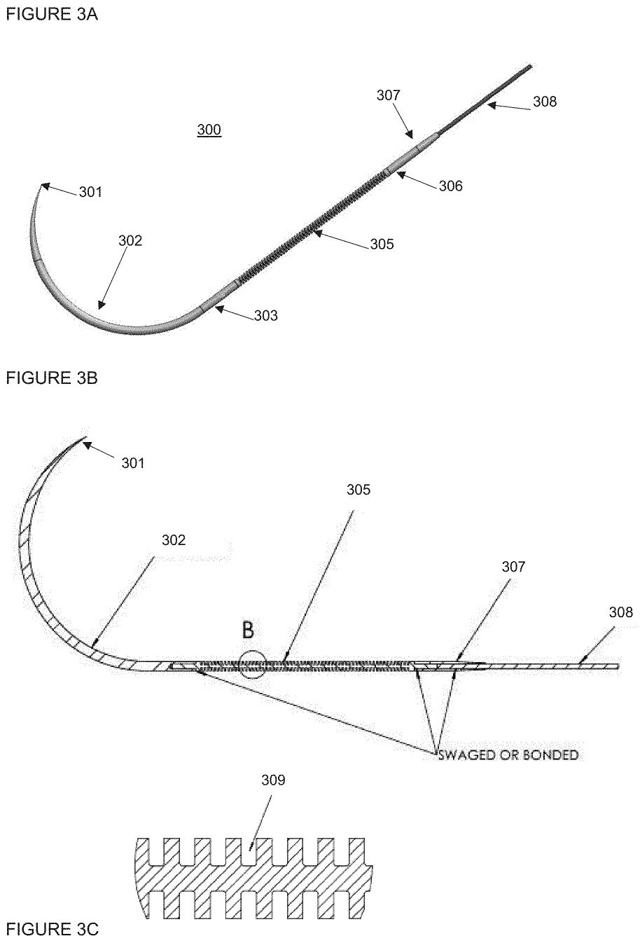

Another exemplary embodiment of the curved suture needle 300 is illustrated in FIG. 3A, a side section view thereof is illustrated in FIG. 3B, and an expanded view of portion B of FIG. 3B is illustrated in FIG. 3C. As illustrated in FIG. 3A and FIG. 3B, the curved suturing needle 300 comprises a tip portion 301 at proximal end of suture needle 300 for piercing a tissue, a needle body 302, the proximal end of which extends from the distal end of the needle tip 301, an extension attachment component 303 the proximal end of which extends from the distal end of the needle body 302, a laser cut wire or molded flexible extension portion 305, the proximal end of which extends from the distal end of the extension attachment component 304, a ramp portion of the flexible extension portion 306 comprising a tapered portion 307, the proximal end of the ramp portion 306 extending from the distal end of the laser cut wire or molded flexible extension portion 305, and a suture thread 308. In this exemplary embodiment, the flexible extension portion 305 is a stainless steel or nickel titanium laser cut wire or an injection molded ire form that is I to form a pattern having alternating projections and indentations. The distance between adjacent projections in the pattern may be constant or may vary. As illustrated in FIG. 3C, a plurality of voids 309 may be formed by the indentations of the pattern in the flexible extension portion 305, and the voids may be filled with an appropriate polymer, such as, for example, various durometer silicones, to allow for a desired flexibility of the curved suture needle. The flexible laser cut wire or molded wire form extension portion 305 is attached to swaged, bonded or crimped at either end to the extension attachment component 303 and the suture thread 308, respectively.

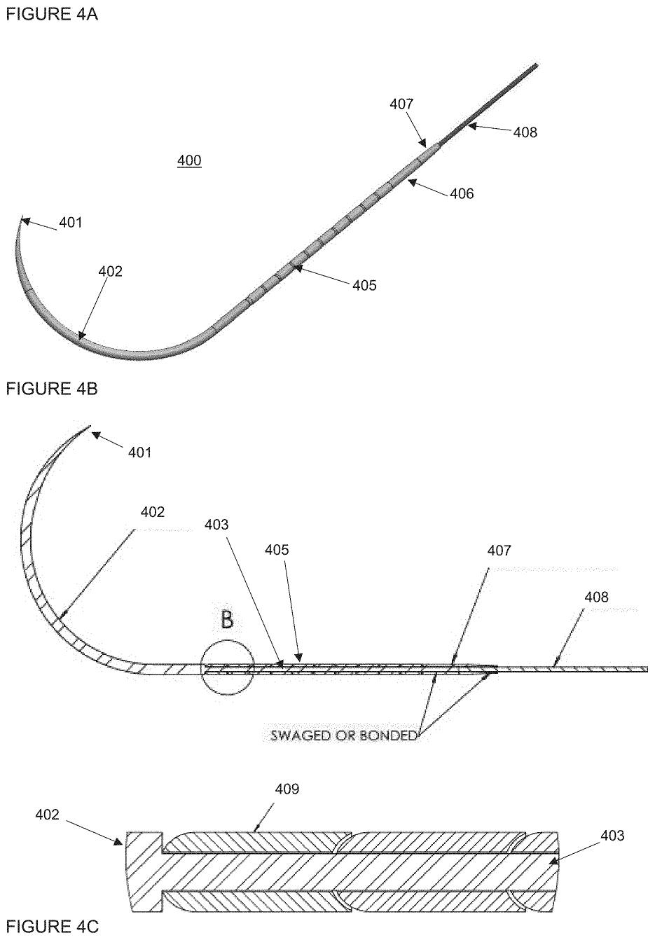

Another exemplary embodiment of the curved suture needle 400 is illustrated in FIG. 4A, a side section view thereof is illustrated in FIG. 4B, and an expanded view of portion B of FIG. 4B is illustrated in FIG. 4C. As illustrated in FIG. 4A and FIG. 4B, the curved suturing needle 400 comprises a tip portion 401 at proximal end of suture needle 400 for piercing a tissue, a needle body 402, the proximal end of which extends from the distal end of the needle tip 401, to a thread attachment component 407 the proximal end of which extends to the distal end of the needle body 402, through a hollow flexible extension portion 405, the proximal end of which extends from a narrow portion of the needle body 403 extending from the needle body 402, a ramp portion of the flexible extension portion 406 comprising a tapered portion 407, the proximal end of the ramp portion 406 extending from the distal end of the flexible extension portion 405, and a suture thread 408. As illustrated in FIG. 4C, the flexible extension portion 405 includes a plurality of stainless steel bullet shaped or conical tubes 409 arranged longitudinally along an outer circumference comprising the flexible extension portion 405. The narrow portion of the needle body 403 provides the internal support for the flexible extension portions 405 shown in this embodiment. Each of the plurality of stainless steel bullet shaped or conical tubes includes one end having a convex shape and an opposite end arranged longitudinally that has a concave shape. The plurality of stainless steel tubes are arranged such that the convex-shaped end of a tube is positioned adjacent to a concave-shaped end of a neighboring tube, as illustrated in FIG. 4C. By virtue of this arraignment, the plurality of stainless steel tubes 409 can form an interlocking structure surrounding the flexible extension portion 405. The flexible extension portion 405 is swaged, bonded or crimped to the tapered distal end of the narrow portion of the needle body 403 at a proximal end of the tapered portion 407 and the suture thread 408 is swaged, bonded or crimped to the distal end of the tapered portion 407. The extension portion 405 is not swaged or bonded to the curved portion of the needle 402 to permit freedom of movement and flexibility.

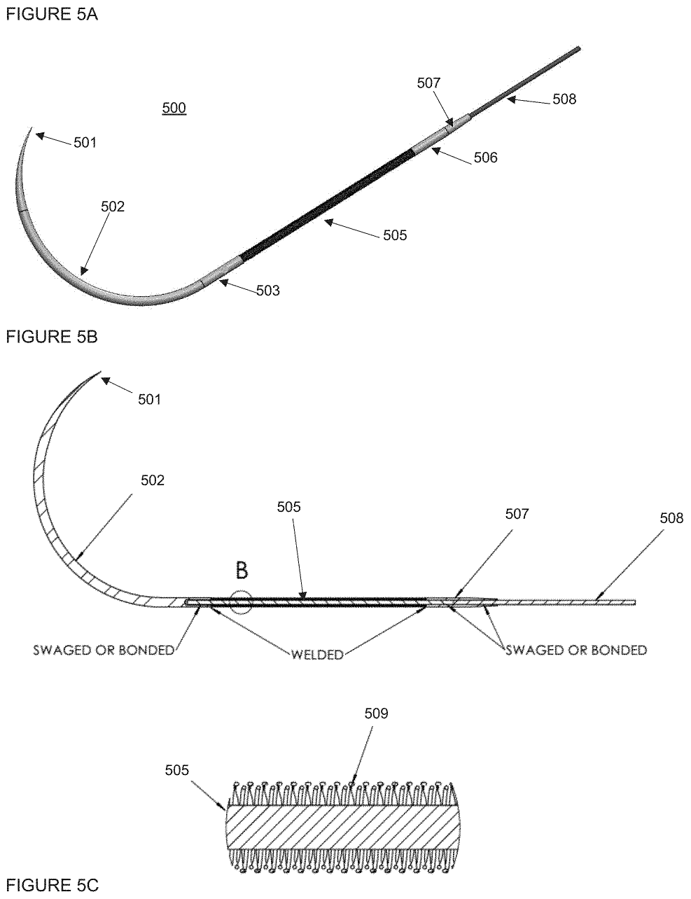

Another exemplary embodiment of the curved suture needle 500 is illustrated in FIG. 5A, a side section view thereof is illustrated in FIG. 5B, and an expanded view of portion B of FIG. 5B is illustrated in FIG. 5C. As illustrated in FIG. 5A and FIG. 5B, the curved suturing needle 500 comprises a tip portion 501 at proximal end of suture needle 500 for piercing a tissue, a needle body 502, the proximal end of which extends from the distal end of the needle tip 501, an internal extension attachment component 503, the proximal end of which extends from the distal end of the needle body 502, a hollow flexible extension portion 505, the proximal end of which extends from the distal end of the internal extension attachment component 503, a ramp portion of the flexible extension portion 506 comprising a tapered portion 507, the proximal end of the ramp portion 506 extending from the distal end of the hollow flexible extension portion 505, and a suture thread 508. In this exemplary embodiment, the flexible extension portion 505 is a stainless steel wire or a metal alloy wire, such as a nickel-titanium alloy wire, surrounded by a braided stainless steel sheath arranged longitudinally along an outer circumference of the flexible extension portion. The distance between adjacent projections in the pattern may be constant or may vary. The flexible hollow extension portion 505 is attached to swaged, bonded or crimped at both ends to the internal extension attachment component 503 and the tapered portion 507, respectively. In exemplary embodiment, the braided sheath 509 may be laser welded to the flexible extension portion 505 at the distal and proximal ends thereof.

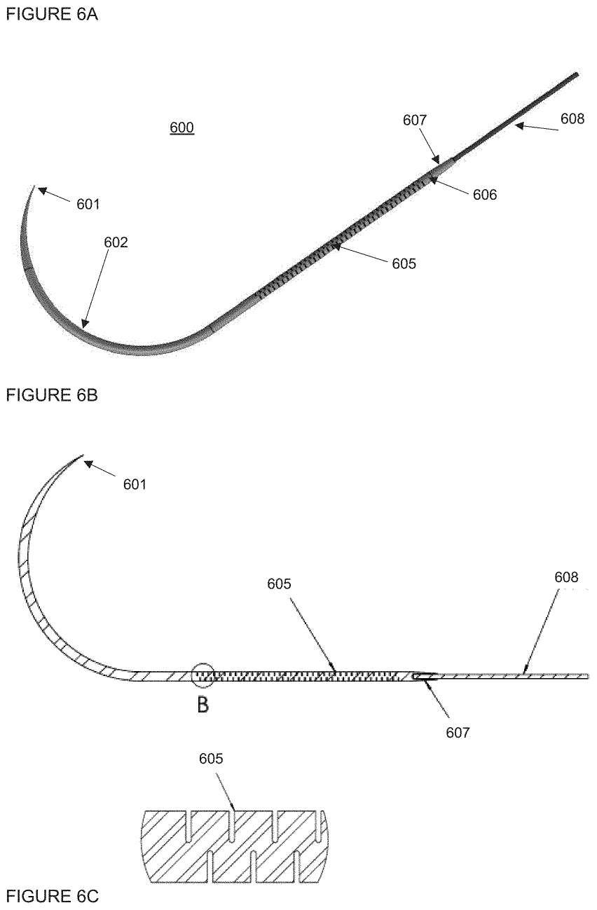

Another exemplary embodiment of the curved suture needle 600 is illustrated in FIG. 6A, a side section view thereof is illustrated in FIG. 6B, and an expanded view of portion B of FIG. 6B is illustrated in FIG. 6C. As illustrated in FIG. 6A and FIG. 6B, the curved suturing needle 600 comprises a tip portion 601 at proximal end of suture needle 600 for piercing a tissue, a needle body 602, the proximal end of which extends from the distal end of the needle tip 601, the proximal end of which extends from the distal end of the needle body 602 to an integral flexible extension portion 605, a ramp portion of the flexible extension portion 606 comprising a tapered portion 607, the proximal end of the ramp portion 606 extending from the distal end of the flexible extension portion 605, and a suture thread 608. As illustrated in FIG. 6C, the flexible extension portion 605 has a structure wherein the outer circumference of the flexible extension portion 605 is laser cut such as a spiral or offset cut as illustrated in FIGS. 6B-6C to create periodically arranged, offset indentations. These laser cuts provide engineered flexure from omni directional in nature (spiral cut) to uni-directional in nature (laser cuts to one side of the flexible extension wire). FIG. 6C illustrates a laser cut pattern to provide bi-directional flexure. The flexible extension portion 605 is connected to the suture thread 608 via a conical swage, or may be bonded or crimped thereto.

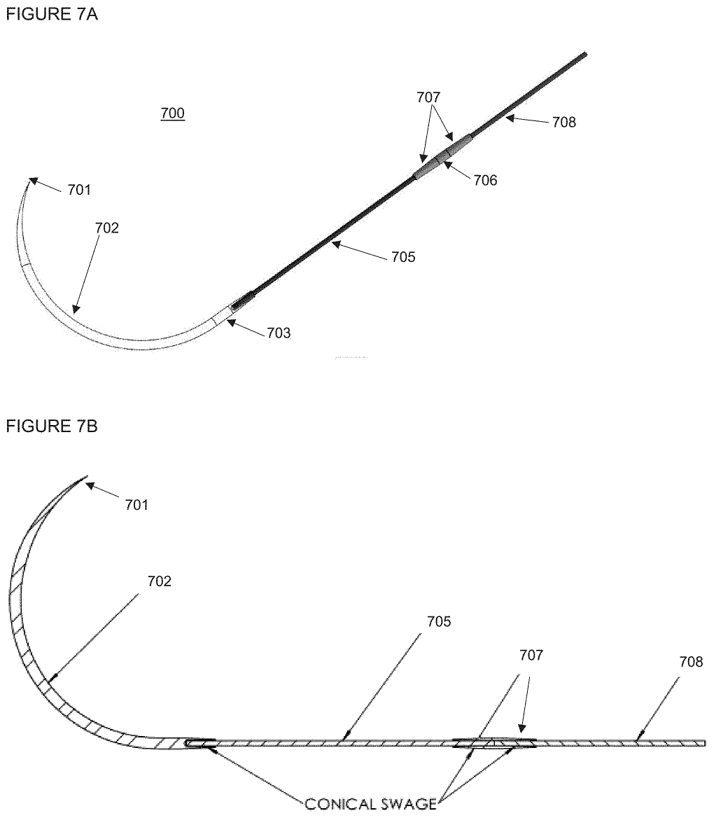

Another exemplary embodiment of the curved suture needle 700 is illustrated in FIG. 7A, and a side section view thereof is illustrated in FIG. 7B. As illustrated in FIG. 7A, the curved suturing needle 700 comprises a tip portion 701 at proximal end of suture needle 700 for piercing a tissue, a needle body 702, the proximal end of which extends from the distal end of the needle tip 701, an extension attachment component 703, the proximal end of which extends from the distal end of the needle body 702, a solid flexible extension portion 705, the proximal end of which extends from the distal end of the extension attachment component 703, a ramp portion of the flexible extension portion 706 comprising a dual tapered portion 707, the proximal end of the ramp portion 706 extending from the distal end of the solid flexible extension portion 705, and a suture thread 708. In this exemplary embodiment, the diameter of the flexible extension portion 705 may be smaller than the diameter of the needle body 702. In such a case, the tapered portion 707 may be a double-ended taper portion, such that the diameter of the tapered portion 707 decreases from a longitudinal center towards the suture thread 708 and the flexible extension portion 705 to enable smooth transition of the roller guide from the suture thread 708 to the needle body 702. The flexible hollow extension portion 705 is connected to the suture thread 708 and/or the needle body 702 via a swage, such as a conical swage, or may be bonded or crimped thereto.

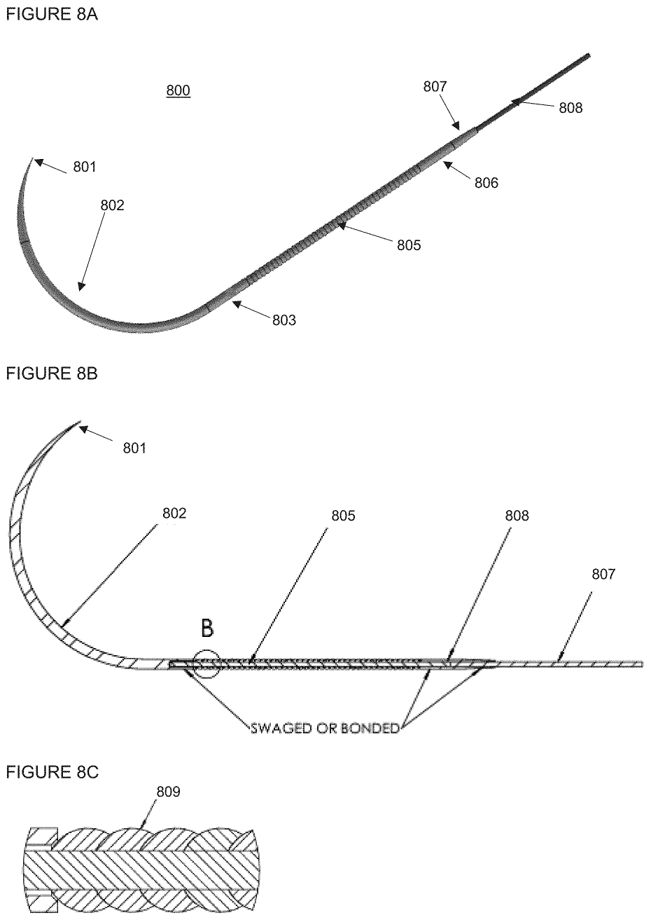

Another exemplary embodiment of the curved suture needle 800 is illustrated in FIG. 8A, a side section view thereof is illustrated in FIG. 8B, and an expanded view of portion B of FIG. 8B is illustrated in FIG. 8C. As illustrated in FIG. 8A, the curved suturing needle 800 comprises a tip portion 801 at proximal end of suture needle 800 for piercing a tissue, a needle body 802, the proximal end of which extends from the distal end of the needle tip 801, an internal extension wire attachment component 803, the proximal end of which extends from the distal end of the needle body 802, a solid flexible extension wire portion 805, the proximal end of which extends from the distal end of the internal extension wire attachment component 803, a ramp portion of the flexible extension portion 806 comprising a tapered portion 807, the proximal end of the ramp portion 806 extending from the distal end of the solid flexible extension wire portion 805, and a suture thread 808. As illustrated in FIG. 8C, the circumference of the flexible extension portion 805 may be surrounded in a longitudinal direction by a layer of stainless steel, nickel titanium or polymer beads 809. A plurality of metal or polymer beads may be arranged along an outer circumference of the solid flexible extension wire portion 805 such that they are nestled against each other. One or more of the plurality of beads 809 may have an oval, an ellipsoid, an oblong, a circular or a semi-circular appearance. One or more of the plurality of beads 809 may have a concave or cup-like portion such that a convex portion of an adjacent bead may be positioned within the concave portion of the bead. The flexible solid extension wire portion 805 may be bonded or crimped to the suture thread 808 and/or the needle body 802. This functions to captivate the plurality of beads between the two crimped sections at 803 and 807 without constraining their movement, to ensure flexibility and unfettered and unlimited movement in any direction.

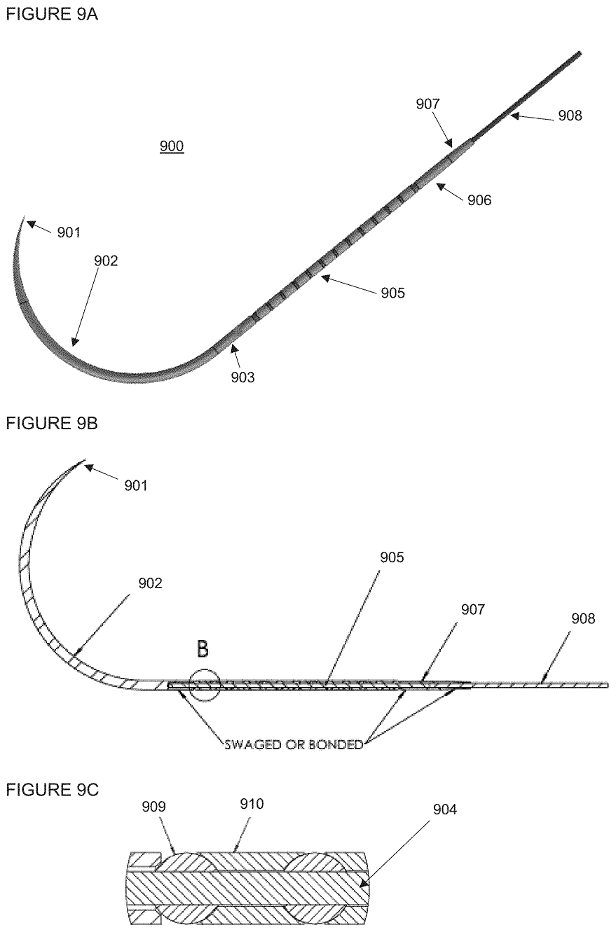

Another exemplary embodiment of the curved suture needle 900 is illustrated in FIG. 9A, a side section view thereof is illustrated in FIG. 9B, and an expanded view of portion B of FIG. 9B is illustrated in FIG. 9C. As illustrated in FIG. 9A, the curved suturing needle 900 comprises a tip portion 901 at proximal end of suture needle 900 for piercing a tissue, a needle body 902, the proximal end of which extends from the distal end of the needle tip 901, an internal extension wire attachment component 903, the proximal end of which extends from the distal end of the needle body 902, a flexible extension portion 905, the proximal end of which extends from the distal end of the internal extension wire attachment component 904, a ramp portion of the flexible extension portion 906 comprising a tapered portion 907, the proximal end of the ramp portion 906 extending from the distal end of the flexible extension portion 905, and a suture thread 908. As illustrated in FIG. 9C, the circumference of the flexible extension portion 905 may include a core portion 904 which may be surrounded in a longitudinal direction by a grouping of stainless steel beads 909 and stainless steel cupped tubes 910. One or more of the plurality of beads 909 may have an oval, an ellipsoid, an oblong, a circular or a semi-circular appearance. One or more of the plurality of tubes 910 may have a concave or cup-like portion at either end of a tube such that a bead 909 may be positioned between two tubes 910. The beads 909 and tubes 910 may be alternate arranged in a pattern to increase flexibility of the flexible extension portion 905. The flexible extension portion 905 may be bonded or crimped to the suture thread 908 and/or the needle body 902. This maintains the flexible groupings of beads 909 and tubing 910 captive between both crimped or swaged ends to ensure flexibility of the extension portion to move in any direction.

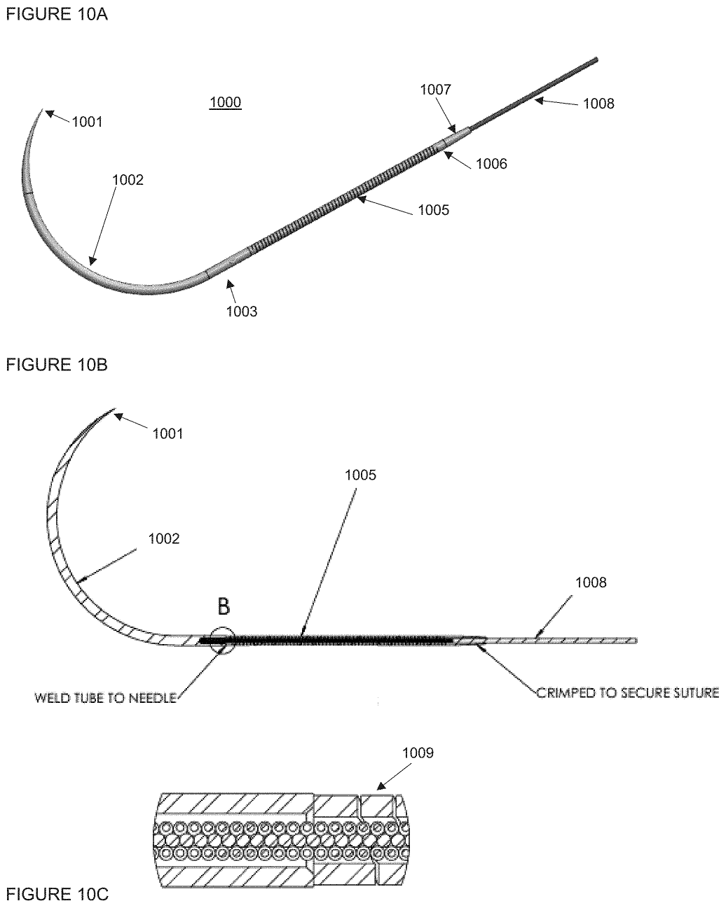

Another exemplary embodiment of the curved suture needle 1000 is illustrated in FIG. 10A, a side section view thereof is illustrated in FIG. 10B, and an expanded view of portion B of FIG. 10B is illustrated in FIG. 10C. As illustrated in FIG. 10A, the curved suturing needle 1000 comprises a tip portion 1001 at proximal end of suture needle 1000 for piercing a tissue, a needle body 1002, the proximal end of which extends from the distal end of the needle tip 1001, a bead acceptance component 1003, the proximal end of which extends from the distal end of the needle body 1002, a hollow flexible extension tubing portion 1005, the proximal end of which extends from the distal end of the bead acceptance component 1003, a ramp portion of the flexible extension portion 1006 comprising a tapered portion 1007, the proximal end of the ramp portion 1006 extending from the distal end of the hollow flexible extension tubing portion 1005, and a suture thread 1008. As illustrated in FIG. 10C, the flexible extension tubing portion 1005 may include stainless steel or nickel titanium tubing either laser cut into numerous patterns or without any laser cuts and include inside to increase flexibility stainless steel or hard polymer microbeads 1009 in an inner portion thereof. In another exemplary embodiment, the flexible extension portion 1005 may include a plurality of microbeads 1009, and the beads may be arranged in any configuration to allow for the passage of the suture thread 1008. The flexible extension tubing portion 1005 may include a single layer or multiple layers of microbeads 1009. The hollow flexible extension tubing portion 1005 may be laser welded to portion 1003 and to portion 1006 and crimped to the suture thread 1008.

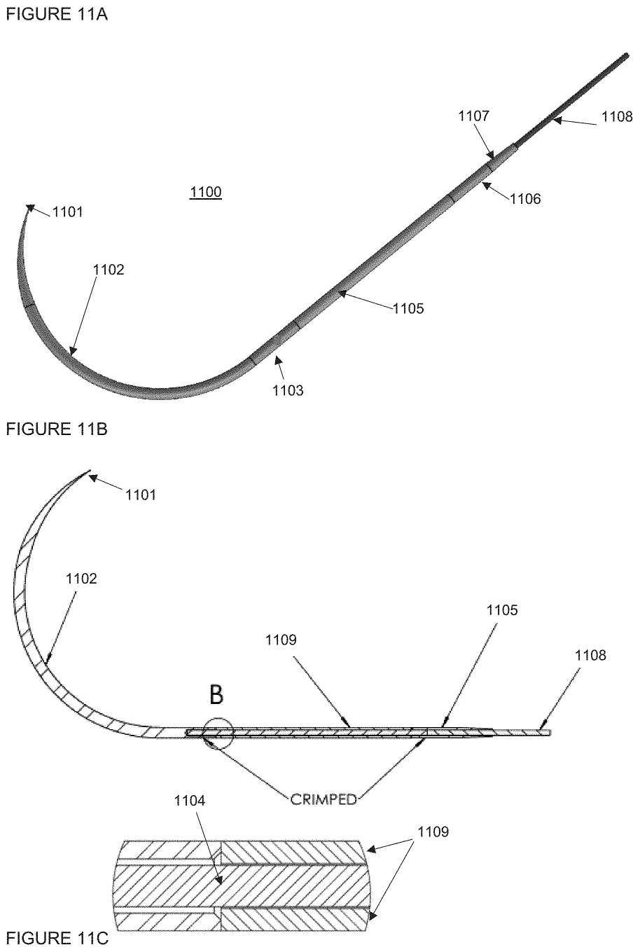

Another exemplary embodiment of the curved suture needle 1100 is illustrated in FIG. 11A, a side section view thereof is illustrated in FIG. 11B, and an expanded view of portion B of FIG. 11B is illustrated in FIG. 11C. As illustrated in FIG. 11A, the curved suturing needle 1100 comprises a tip portion 1101 at proximal end of suture needle 1100 for piercing a tissue, a needle body 1102, the proximal end of which extends from the distal end of the needle tip 1101, an extension wire attachment component 1103, the proximal end of which extends from the distal end of the needle body 1102, a solid internal flexible extension wire portion 1105, the proximal end of which extends from the distal end of the thread attachment component 1103, a ramp portion of the flexible extension portion 1106 comprising a tapered portion 1107, the proximal end of the ramp portion 1106 extending from the distal end of the hollow flexible extension external tubing portion 1109, and a suture thread 1108. As illustrated in FIG. 11B and FIG. 11C, the circumference of the flexible extension portion 1105 may include a core portion 1104 which may be surrounded by a flexible polymer sleeve 1109. The core portion 1104 may be bonded or crimped or swaged to the suture taper portion 1107 and/or the extension wire attachment component 1103.

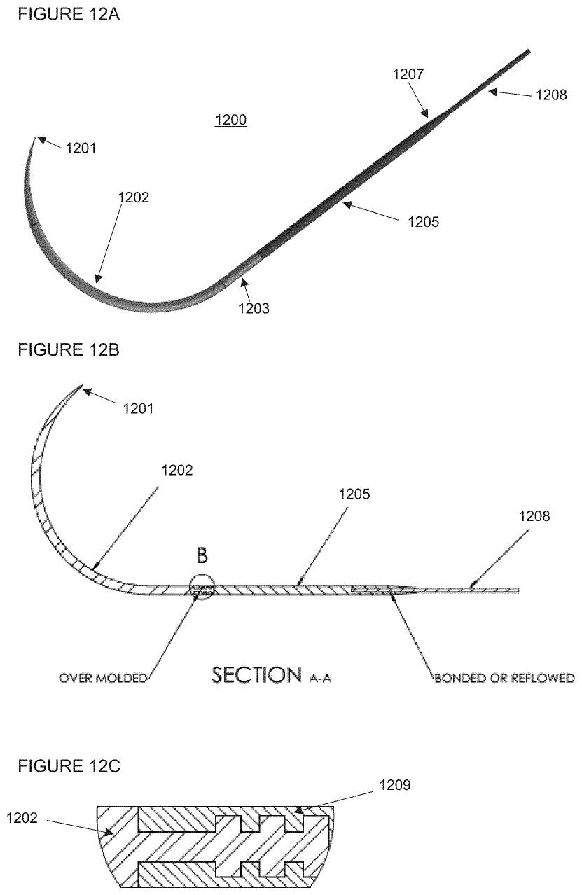

Another exemplary embodiment of the curved suture needle 1200 is illustrated in FIG. 12A, a side section view thereof is illustrated in FIG. 12B, and an expanded view of portion B of FIG. 12B is illustrated in FIG. 12C. As illustrated in FIG. 12A, the curved suturing needle 1200 comprises a tip portion 1201 at proximal end of suture needle 1200 for piercing a tissue, a needle body 1202, the proximal end of which extends from the distal end of the needle tip 1201, a machined undercut feature component 1203, the proximal end of which extends from the distal end of the needle body 1202, an insert molded solid flexible extension portion 1205, the proximal end of which extends from the distal end of the undercut feature attachment component 1203, a ramp portion comprising a tapered portion 1207, the proximal end of the ramp portion 1207 extending from and as part of the distal end of the solid flexible extension portion 1205, and a suture thread 1208. As illustrated in FIG. 12C, the circumference of the flexible extension portion 1205 is the same as that of the needle body 1202 and may be formed by injection molding a polymer exterior layer 1209 onto the end of the needle body 1202. The ramp portion comprising the tapered portion 1207 may be formed continuously with the flexible extension portion 1205 or may be separately formed. The insert molded solid flexible extension portion 1205 with the exterior layer 1209 may be bonded or crimped to the suture thread 1208 and/or the needle body 1202.

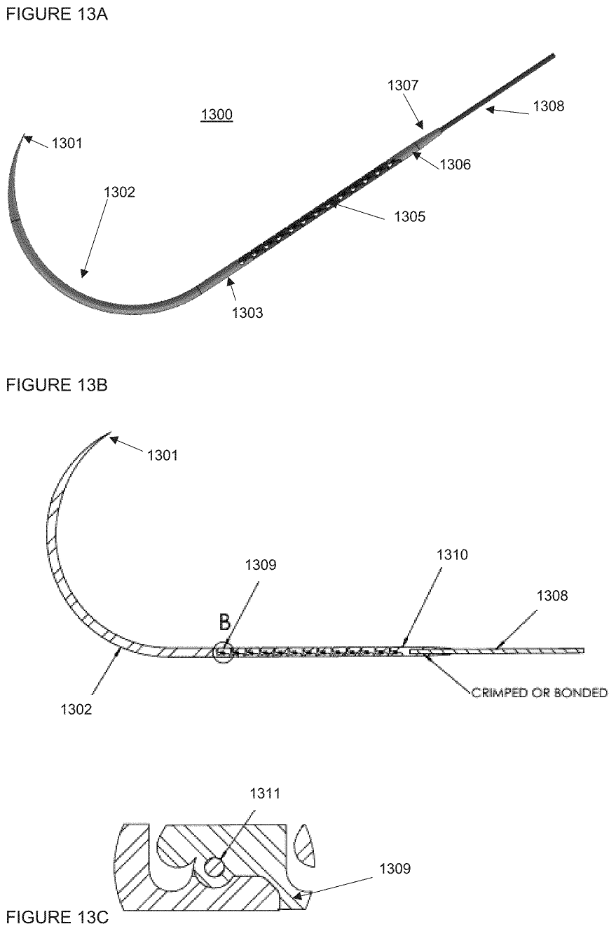

Another exemplary embodiment of the curved suture needle 1300 is illustrated in FIG. 13A, a side section view thereof is illustrated in FIG. 13B, and an expanded view of portion B of FIG. 13B is illustrated in FIG. 13C. As illustrated in FIG. 13A, the curved suturing needle 1300 comprises a tip portion 1301 at proximal end of suture needle 1300 for piercing a tissue, a needle body 1302, the proximal end of which extends from the distal end of the needle tip 1301, a chain link attachment component 1303, the proximal end of which extends from the distal end of the needle body 1302, a flexible extension portion 1305, the proximal end of which extends from the distal end of the chain link attachment component 1303, a ramp portion of the flexible extension portion 1306 comprising a tapered portion 1307, the proximal end of the ramp portion 1307 extending from the distal end of the flexible extension portion 1305, and a suture thread 1308. As illustrated in FIG. 13B and FIG. 13C, the flexible extension portion 1305 can include a chain link connection portion 1309 connecting the needle body 1302 to the suture thread 1308. The chain link connection portion 1309 can include a tapered end link 1310, which combines the ramp portion 1306 and a tapered portion 1307 in a single feature. The tapered end link 1310 may be formed continuously with the chain link connection portion 1309 or may be separately formed. As illustrated in FIG. 13C, the chain link connecting portion 1309 can include pivot pins 1311 connecting a plurality of links to form the chain link connecting portion 1309. The flexible extension portion 1305 may be bonded or crimped to the suture thread 1308 and laser welded to the needle body 1202.

Another exemplary embodiment of the curved suture needle 1400 is illustrated in FIG. 14A, a side section view thereof is illustrated in FIG. 14B. As illustrated in FIG. 14A, the curved suturing needle 1400 comprises a tip portion 1401 at proximal end of suture needle 1400 for piercing a tissue, a needle body 1402, the proximal end of which extends from the distal end of the needle tip 1401, a polymer injection port 1403, the proximal end of which extends from the distal end of the needle body 1402, a hollow flexible extension portion 1405, the proximal end of which extends from the distal end of the needle body 1402, a tapered portion 1407, the proximal end of the tapered portion 1407 extending from the distal end of the hollow flexible extension portion 1405, and a suture thread 1408. As illustrated in FIG. 14B and FIG. 14C, the flexible extension portion 1405 includes a laser-cut nickel titanium or stainless steel tube, which is filled inside with a polymer filling. A short rear portion of the flexible extension portion 1405 is hollow to allow movement and/or passage and securement of the suture thread 1408. The hollow flexible extension portion 1405 may be bonded or crimped to the suture thread 1408 and/or the needle body 1402. Alternatively, the distal end of the needle body 1402 may be laser welded to the flexible extension portion 1405.

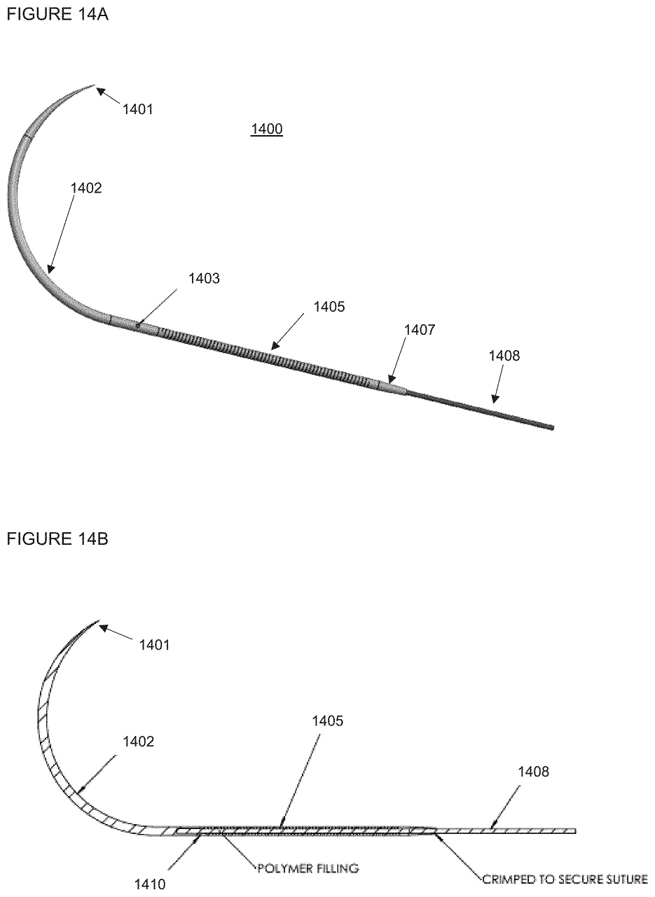

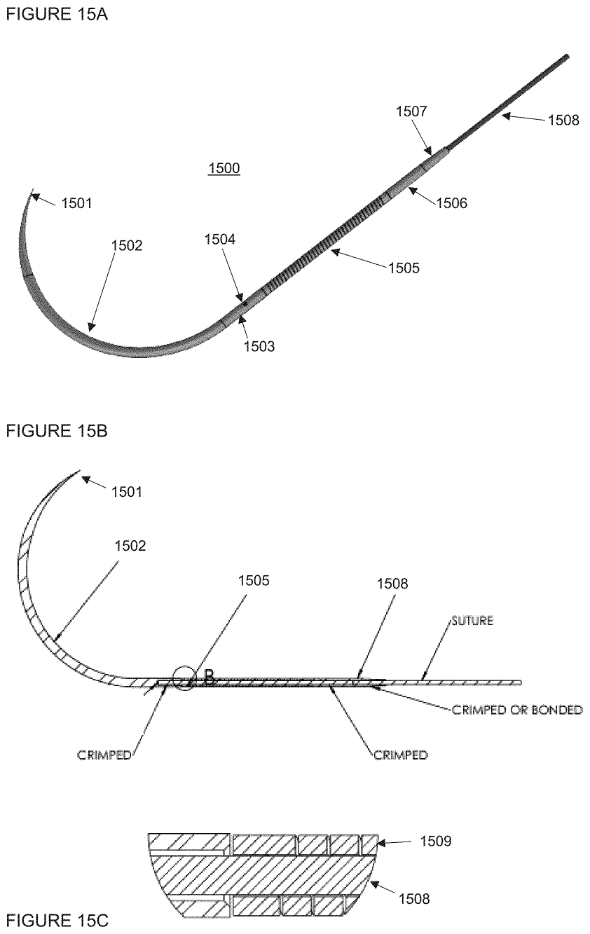

Another exemplary embodiment of the curved suture needle 1500 is illustrated in FIG. 15A, a side section view thereof is illustrated in FIG. 15B, and an expanded view of portion B of FIG. 15B is illustrated in FIG. 15C. As illustrated in FIG. 15A, the curved suturing needle 1500 comprises a tip portion 1501 at proximal end of suture needle 1500 for piercing a tissue, a needle body 1502, the proximal end of which extends from the distal end of the needle tip 1501, a thread attachment component 1503, with a viewing port 1504, the proximal end of which extends from the distal end of the needle body 1502, a flexible extension portion 1505, the proximal end of which extends from the distal end of the thread attachment component 1504, a ramp portion of the flexible extension portion 1506 comprising a tapered portion 1507, the proximal end of the ramp portion 1506 extending from the distal end of the flexible extension portion 1505, and a suture thread 1508. As illustrated in FIG. 15C, the flexible extension portion 1505 may include a stainless steel laser cut outer sleeve 1509 positioned over the suture thread 1508 which is crimped at portion 1503. The hollow flexible extension portion of 1505 may be bonded or crimped to the suture thread 1508 and/or laser welded to the needle body 1502 at a single position or at multiple positions. In another embodiment, the laser cut sleeve 1509 may be crimped to be in a tapered shape such that the diameter of the last cut sleeve 1509 decreases towards the proximal end of the suture body 1508. In another embodiment, the tapered end of the laser cut sleeve 1509 may replace the proximal end of the ramp portion 1506 and/or the tapered portion 1507.

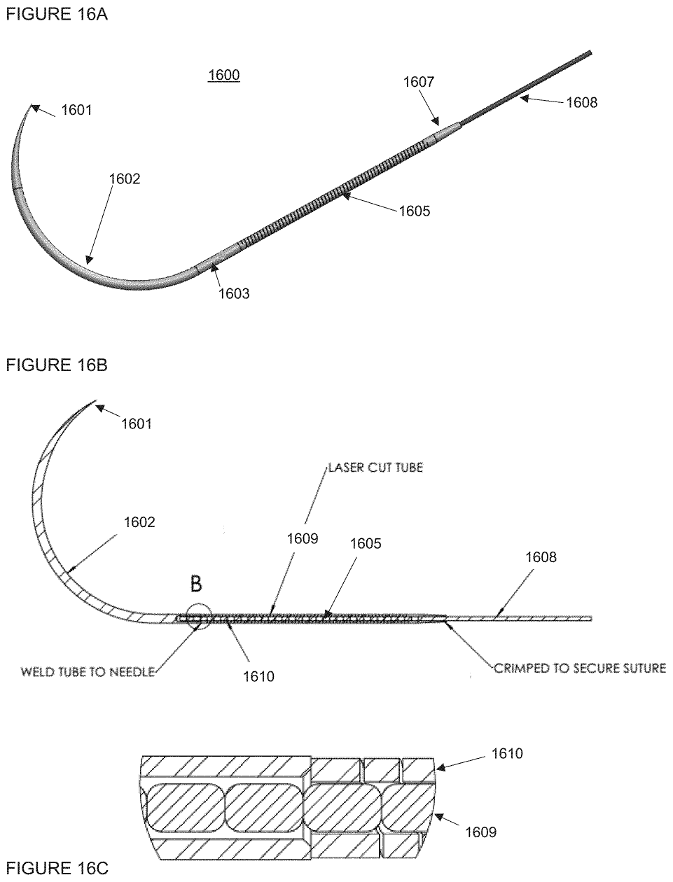

Another exemplary embodiment of the curved suture needle 1600 is illustrated in FIG. 16A, a side section view thereof is illustrated in FIG. 16B, and an expanded view of portion B of FIG. 16B is illustrated in FIG. 16C. As illustrated in FIG. 16A, the curved suturing needle 1600 comprises a tip portion 1601 at proximal end of suture needle 1600 for piercing a tissue, a needle body 1602, the proximal end of which extends from the distal end of the needle tip 1601, a bead engagement portion 1603, the proximal end of which extends from the distal end of the needle body 1602, a flexible extension portion 1605, the proximal end of which extends from the distal end of the bead engagement portion 1603, a tapered portion 1607 of the flexible extension portion 1605, the proximal end of the tapered portion 1607 extending from the distal end of the hollow flexible extension portion 1605, and a suture thread 1608. As illustrated in FIG. 16B and FIG. 16C, the hollow flexible extension portion 1605 may include a stainless steel laser cut tube or nickel titanium sleeve 1609 positioned on an outer surface along a longitudinal axis of the hollow flexible extension portion 1605 that is laser welded or crimped to bead engagement portion 1603 of the needle 1602. The hollow flexible extension portion 1605 may include polymer or metal pellets 1610 as a filling on an inside portion thereof. The hollow flexible extension portion 1605 may be bonded or crimped to the suture thread 1608 and/or the needle body 1602 at a single position or at multiple positions. In another embodiment, the laser cut sleeve 1609 may be crimped to be in a tapered shape such that the diameter of the last cut sleeve 1609 decreases towards the proximal end of the suture body 1608. In another embodiment, the tapered end of the laser cut sleeve 1609 may replace the proximal end of the ramp portion 1606 and/or the tapered portion 1607.

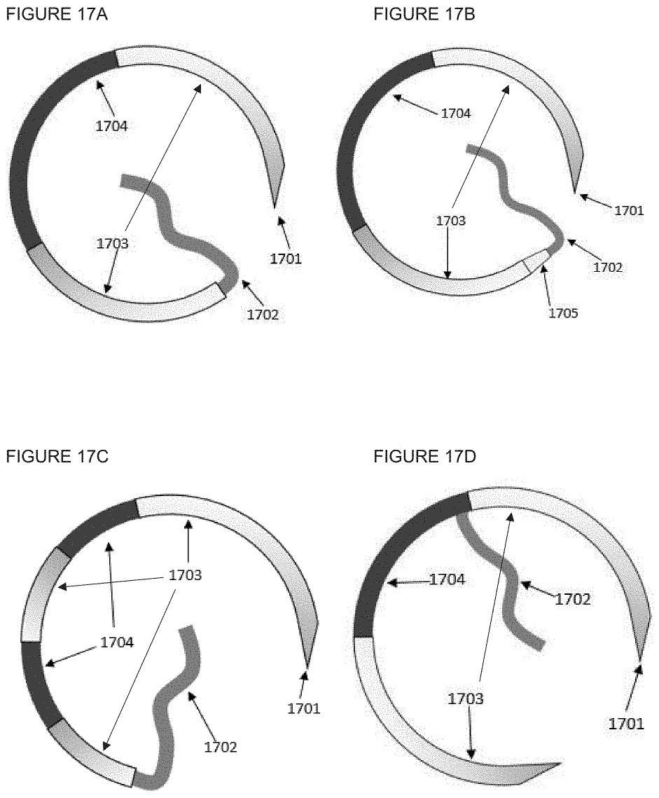

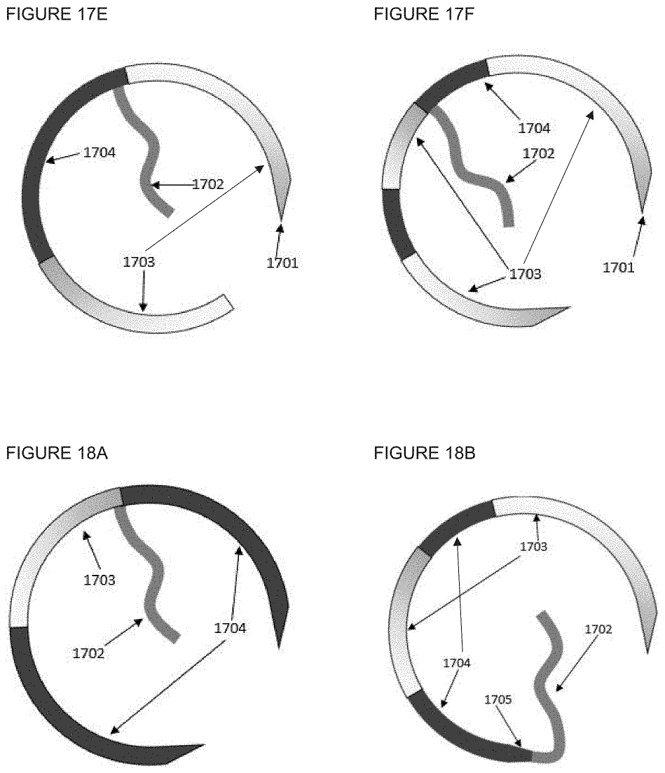

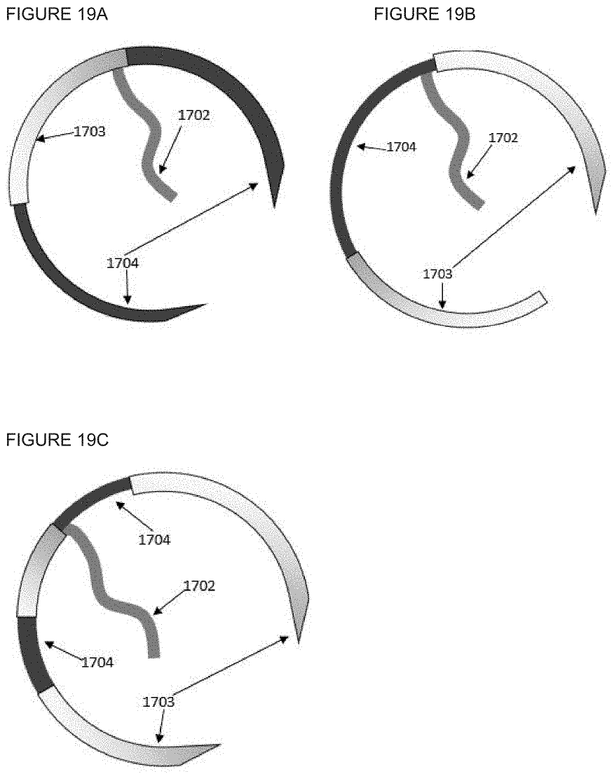

Another exemplary embodiment of the curved suture needle is illustrated in FIGS. 17A-17C, each of which is a side view of a curved suture needle. The curved suture needle of this exemplary embodiment includes a needle body including non-flexible sections 1703 and flexible sections 1704 in one or more combinations. The curved suture needle includes a tip portion 1701 at a proximal end of the suture needle for piercing a tissue and a needle body extending from the distal end of the needle tip 1701 and including the flexible section 1704 and the non-flexible sections 1703. In certain embodiments, the non-flexible sections 1703 are separated by the flexible sections 1704. In certain other embodiments, the flexible sections 1704 are separated by non-flexible sections 1703. A plurality of non-flexible sections 1703 can be separated by a single flexible section 1704 (FIG. 17A) and a plurality of non-flexible sections 1703 can be separated by a plurality of flexible sections 1704 (FIG. 17C). As illustrated in FIGS. 19A-19C, when a curved suture needle includes multiple flexible sections 1704 or non-flexible sections 1703, any or both of the tip portions may be part of a flexible portion (FIG. 19A) or a non-flexible section (FIG. 19C). In certain other embodiments, the curved suture needle can further include a tapered suture attachment section 1705 (FIGS. 17B and 18B), but is not limited thereto.

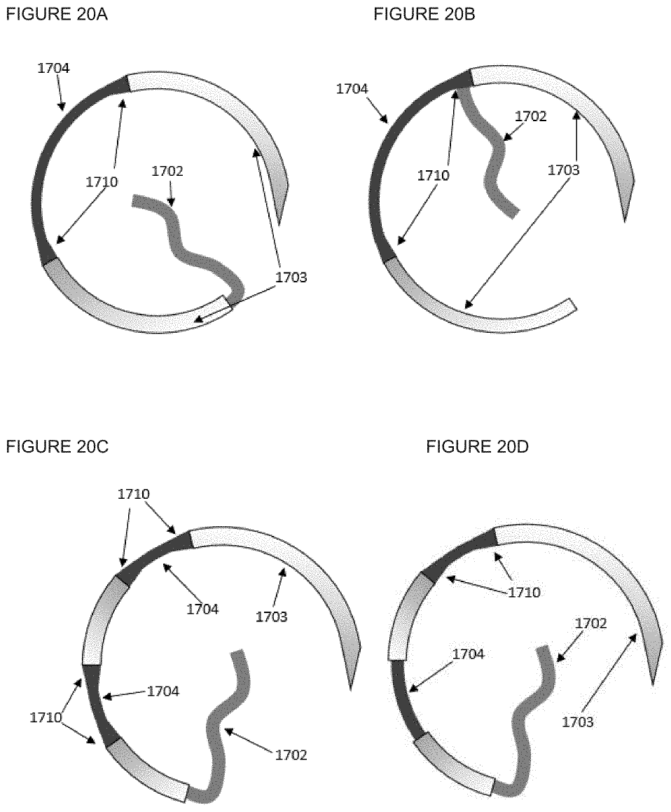

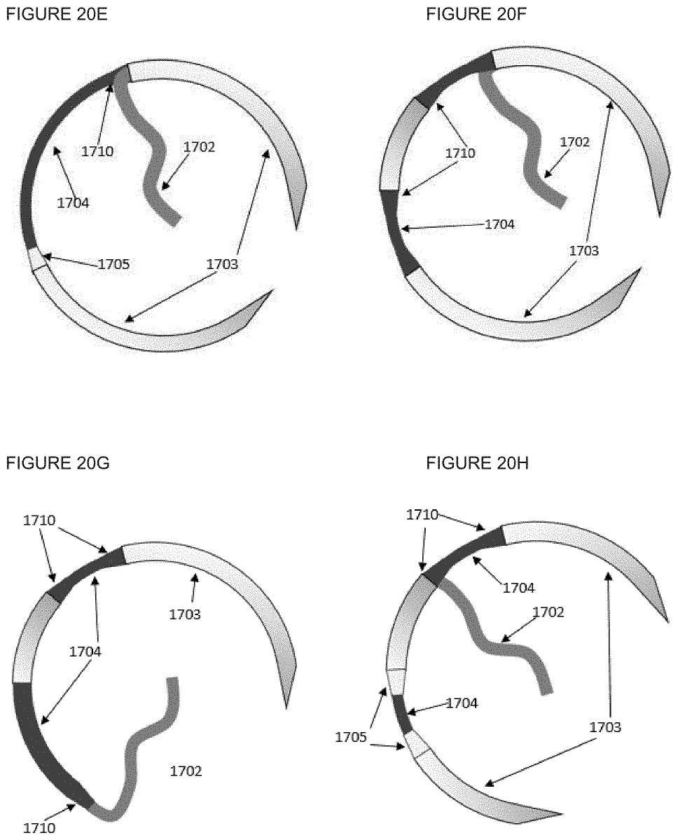

The flexible section(s) 1704 could be crimped, welded, adhered or otherwise attached by any joining method or process to the adjacent non-flexible sections of the needle. The diameter of the flexible sections 1704 can be the same as or different from the diameter of the non-flexible sections 1703. When the diameters of the non-flexible section 1703 and flexible sections 1704 are different, the diameter of the flexible sections 1704 can be smaller or larger than the diameter of the non-flexible sections 1703. When there are multiple intervening flexible and/or non-flexible sections at least some of the flexible and non-flexible sections can have the same diameter and at least some of the flexible and non-flexible sections can have different diameters, and the suture needle can include a combination of different sections having varying diameters. As illustrated in FIG. 19B, a certain exemplary embodiment, the curved suture needle can include at least three sections having two non-flexible section 1703 and one flexible section 1704 each with its own diameter different from each of the other sections. In embodiments where adjacent sections have different diameters the crimping (or any other attachment method used for joining the sections) can be on-center (FIGS. 19A-19B) or off-center (FIG. 19C). Off-center attachment is shown in conjunction with crimping of the suture to a non-flexible section in FIG. 19C, but the method of attachment is not limited thereto, as described elsewhere in the present disclosure. When the diameter of adjacent flexible and/or non-flexible sections are different, a transition portion between the flexible and/or non-flexible sections having different diameters can be tapered (FIGS. 20A-20C showing tapered transitions 1710 between two diameters (FIG. 20A), three diameters (FIG. 20B) and multiple sections with tapered transitions between different diameters (FIG. 20C)) or non-tapered (FIGS. 19B-19C) or a combination of the same (FIGS. 20D-20I).

As illustrated in FIGS. 17D-17F, the position of the suture 1702 is not limited to the swage ends of the curved suture needle, and can be attached to other sections of the needle that are not the usual swage end of the needle. In certain exemplary embodiments, one or both swage ends of the curved suture needle can be tipped, such that the one or both swage ends are capable of piercing tissue. In certain exemplary embodiments, one or both swage ends of the curved suture needle may be a part of the non-flexible sections 1703 (FIGS. 17A-17C) and/or one or both swage ends of the curved suture needle may be a part of the flexible portions 1704 (FIG. 18B). In certain embodiments in which the suture attachment portion is tapered, this tapered suture attachment portion may be part of a flexible section (FIG. 17B) or a non-flexible section (FIG. 18B).

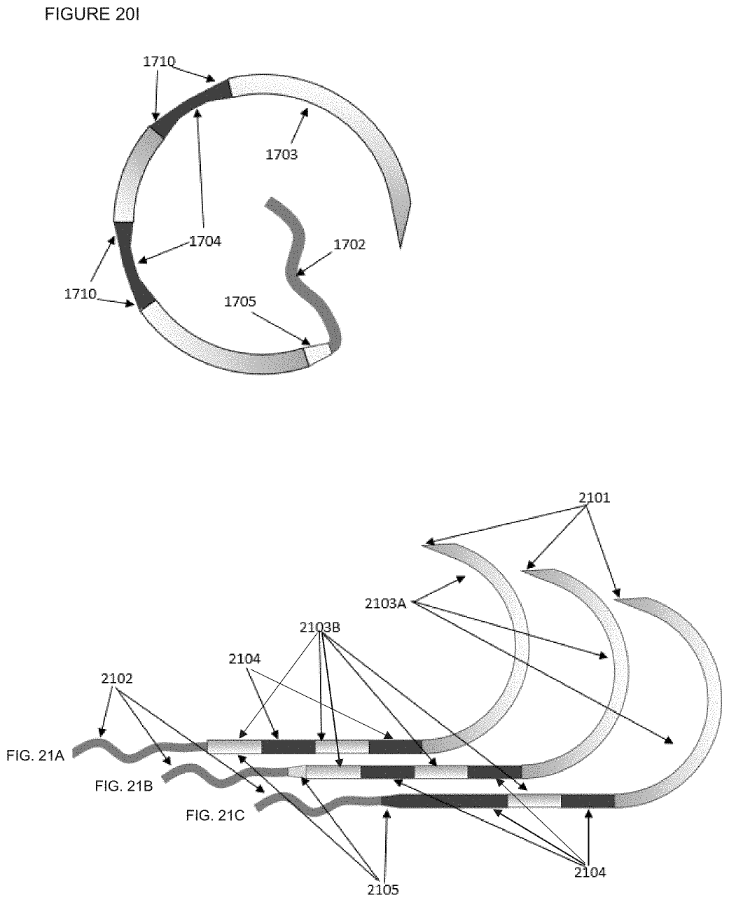

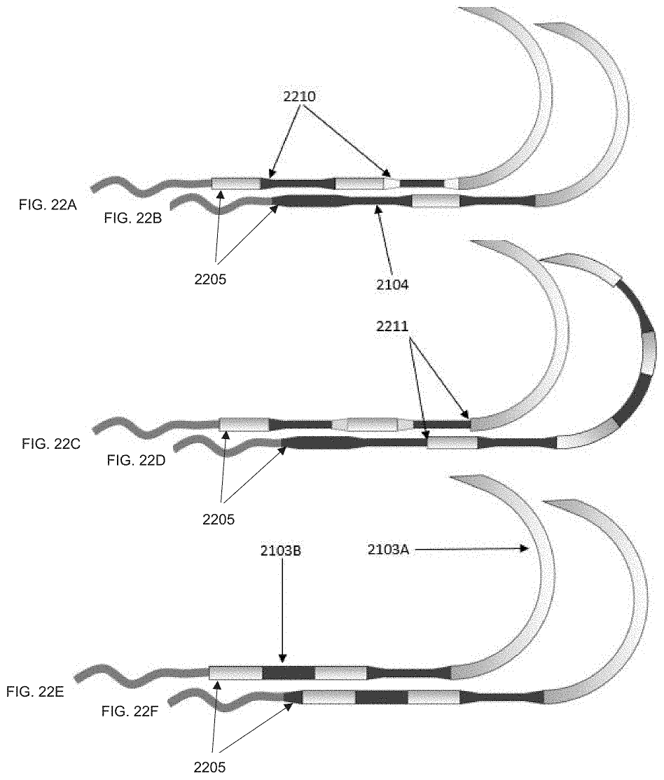

Another exemplary embodiment of the present disclosure is a curved suture needle including a curved section and a plurality of straight sections, as illustrated in FIGS. 21A-21C, 22A-22F. The suture needle can include at least one non-flexible curved section, one or more flexible sections, and one or more non-flexible straight sections, including any combination thereof such as a plurality of flexible straight sections in combination with a plurality of non-flexible straight sections, a plurality of flexible straight sections in combination with one non-flexible straight section, one flexible straight section in combination with a plurality of non-flexible straight sections, and the like, and where a plurality denotes two or more sections.

FIGS. 21A-21C illustrate a curved suture needle having a non-flexible curved section 2103A combined with at least one flexible straight section 2104 and one non-flexible straight section 2103B. The curved suture needle includes a tip portion 2101 at a proximal end of the suture needle for piercing a tissue and a needle body extending from the distal end of the needle tip 2101 and including the flexible and non-flexible sections 2104, 2103A and 2103B. As shown in FIGS. 21A-21C, the suture needle can also include a suture attachment portion 2105 that can be non-tapered (FIG. 21A) or tapered (FIG. 21B). The tapered suture attachment portion 2105 can be part of a of non-flexible straight section 2103B (FIG. 21B) or a flexible straight section 2104 (FIG. 21C).

As shown in FIGS. 22A-22F, the suture needles can have a combination of flexible and non-flexible sections having different diameters. When the diameter of adjacent flexible and/or non-flexible sections are different a transition portion between the flexible and/or non-flexible sections having different diameters can be a tapered transition section 2210, a non-tapered transition section 2211, or a combination of tapered transition sections 2210 and non-tapered transition sections 2211, as shown in FIGS. 22C-22D. Tapered and non-tapered suture attachment portions 2205 are also shown in FIGS. 22A-22F. Tapered suture attachment portions 2205 can be part of a non-flexible section or a flexible section. Features of FIGS. 22A-22F in common with FIGS. 21A-21C are not repeated herein for brevity.

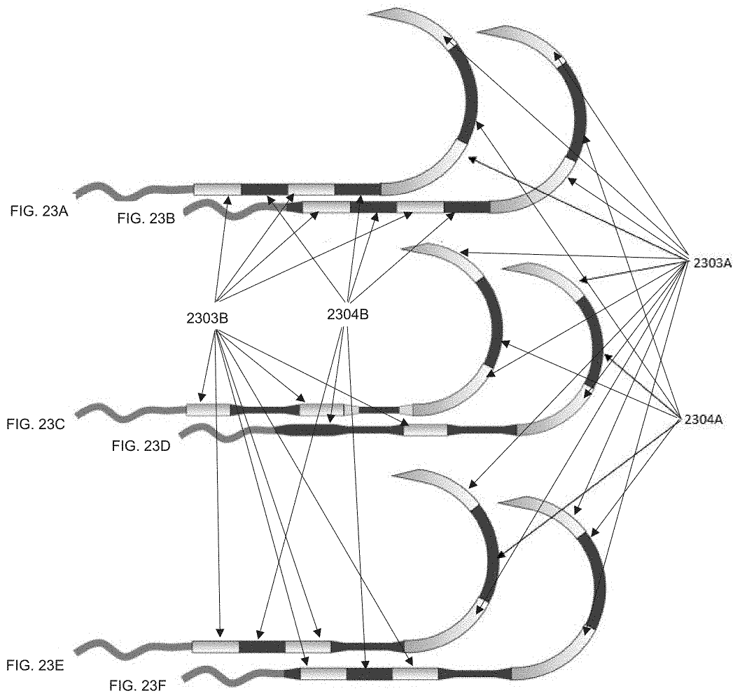

As shown in FIGS. 23A-23F, a curved suture needle can include one or more non-flexible curved sections 2303A combined with one or more flexible curved sections 2304A, one or more non-flexible straight section 2303B, one or more flexible straight sections 2304B, and any combination thereof. As shown in FIGS. 23A-23F, a flexible curved section 2304A may be positioned between two non-flexible sections 2303A, but the curved suture needle is not limited thereto, and all combinations of straight and curved sections are contemplated as being within the scope of the present disclosure. Features of FIGS. 23A-23F in common with other drawings herein are not repeated for brevity.

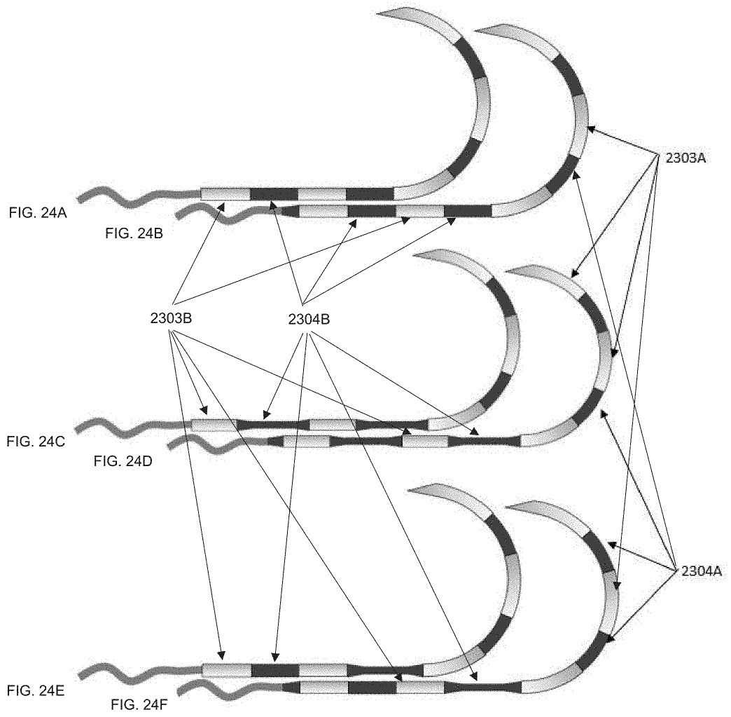

FIGS. 24A-24F illustrate certain other embodiments of the present disclosure. As shown in FIGS. 24A-24F, the suture needle can include a plurality of flexible curved sections 2304A alternating with a plurality of non-flexible curved sections 2303A. Also as shown in FIGS. 24A-24F, the suture needle can include a plurality of flexible straight sections 2304B alternating with a plurality of non-flexible straight sections 2303B. Other features of FIGS. 24A-24F in common with other drawings herein are not repeated for brevity.

FIGS. 25A-25B are illustrations of certain exemplary embodiments of the present disclosure showing a combination of one or more flexible curved sections 2304A, flexible straight sections 2304B, non-flexible curved sections 2303A, and non-flexible straight sections 2303B. The present disclosure contemplates any such combinations, such as a plurality of flexible straight sections 2304B in combination with a plurality of non-flexible straight sections 2303B, a plurality of flexible straight sections 2304B in combination with one non-flexible straight section 2303B, one flexible straight section 2304B in combination with a plurality of non-flexible straight sections 2303B, a plurality of flexible curved sections 2304A in combination with one non-flexible curved section 2303A, one flexible curved section 2304A in combination with a plurality of non-flexible curved sections 2303A, a plurality of flexible curved sections 2304A in combination with a plurality of non-flexible curved sections 2303A, and the like, and any combinations thereof, where a plurality denotes two or more sections. The various straight and curved sections can have the same or different diameters, and such various straight and curved sections can be connected via tapered and/or non-tapered connection sections, as described above. Common features of the drawings of the present disclosure are omitted for brevity.

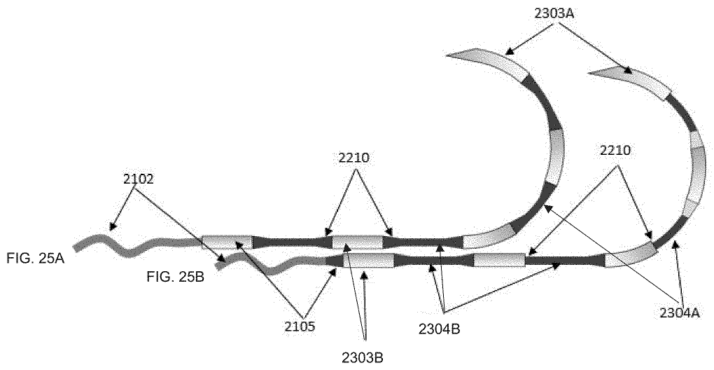

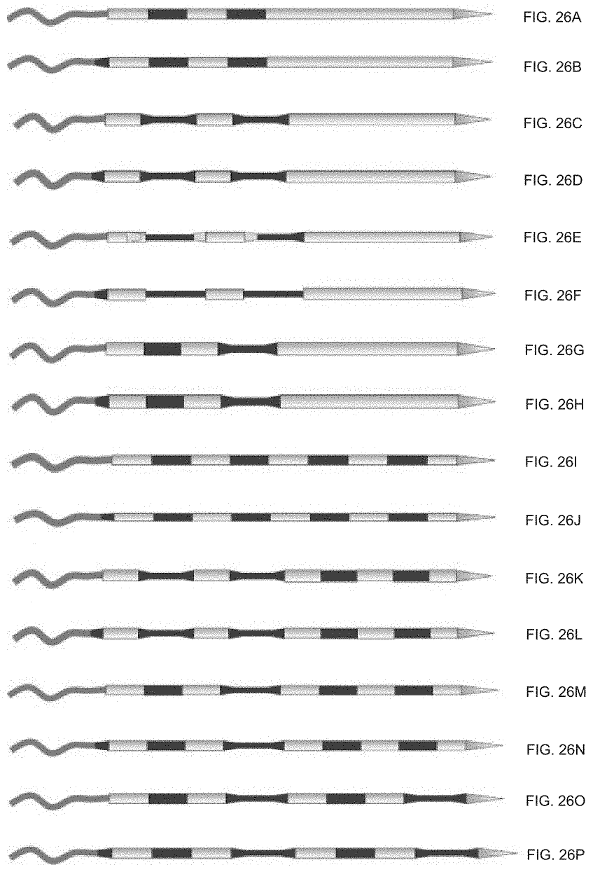

FIGS. 26A-26P illustrate various exemplary embodiments of a non-curved needle including a tip portion at a proximal end of the suture needle for piercing a tissue and a needle body extending from the distal end of the needle tip and including one or more non-curved flexible sections and one or more non-curved non-flexible sections, and any combinations thereof, as described above. These embodiments include other features described above, such as sections having varying diameters, tapered and non-tapered connection portions, and each of these sections can be part of a flexible or a non-flexible section, as described herein. Overlapping descriptions are omitted herewith for brevity.

The texture, finish, cross-section, geometry or processes used to achieve any desired characteristic of the flexible section(s) described above can be the same as the non-flexible sections, as known in the art and/or described herein. Certain exemplary embodiments of the present disclosure describe a curved shape of the flexible and/or non-flexible sections, but the shape is not limited thereto, and can include non-curved structures, such as straight, J-shaped, S-shaped, U-shaped, angled, spiral, compound curved, zigzag, complex tridimensional shape, and the like, and any combinations thereof. The length of the flexible and non-flexible sections of the curved suture needle are not limited, and any of the non-flexible sections and flexible sections can be of any length and can be in any proportion with any other flexible or non-flexible section.

These and other embodiments of the curved suturing needle create a procedural benefit and efficiency by loading the needle onto a needle driver to a position ready for suturing all in a single step. The rollers may be closed over the suture material and may rotate in opposite directions (x1 and x2, respectively) to advance the needle forward. As the needle advances between the rollers of the needle driver, the rollers smoothly advance the needle from the suture material to the tapered portion of the ramp portion. Without opening the rollers, the rollers may continue to rotate to advance the needle along the tapered portion over the ramp portion.

In the absence of the tapered portion, this would represent a step or lip on the needle that may be damaged by the rollers, or conversely, that may damage the rollers. Accordingly, in the absence of the tapered portion, the clinician would have to use a grasper to hold/stabilize the needle, then open the needle driver to release the suture it was grasping during needle insertion and then grasp the needle with the needle driver once positioned over the needle body in preparation for suturing. In a laparoscopic setting each of these steps may require time, effort, coordination and maybe even repetition due to the lack of depth perception (2D visualization systems used mostly) and decreased dexterity, fulcrum effect of port, etc. Finally, the rotation of the rollers advances the needle so that it is positioned between the rollers and properly loaded for suturing without ever opening the rollers. These motions may be reversed to unload the needle, such as to remove the needle from a suture site (e.g., from the abdomen or from a laparoscopic surgical site).

The material configuring the suture needle is not limited, and may comprise a metal or a metal alloy, such as, for example, a biocompatible metal or metal alloy. In certain embodiments, the suture needle comprises any of steel wire, a martensitic stainless steel, or an austenitic stainless steel. In certain embodiments, the suture needle is comprised of a magnetic or magnetizable material. When formed of steel wire or martensitic stainless steel, the needle may be hardened by thermal treatment. Other processes when forming or finishing the needle may be siliconization, coating with any number or combination of biocompatible coating materials or lubrication with any number or combination of biocompatible lubricating agents, among other processes. As illustrated in the drawings, for example, the suture thread may be swaged to the needle by the thread attachment component. Accordingly, the needle is generally an atraumatic needle, i.e., eyeless needle, having a suture material or thread attached at an end by swaging whereby the suture material is inserted into a channel at the blunt end of the needle, such as into the thread attachment component, which is then deformed to a final shape to hold the suture and needle together. The needle may be permanently swaged to the suture material or may be designed to come off the suture material with a sharp straight tug. These “pop-offs” are commonly used for interrupted sutures, where each suture is only passed once and then tied.

The material of the flexible extension component of the suture needle may be any suitable material including a metal, a metal alloy, a polymer and the like. The metal may be selected from any suitable metal, including nickel, titanium, aluminum, chromium, iron, and alloy thereof, including alloys with other metals and non-metals. The material of the flexible extension component can include stainless steel. The polymer may be selected from polyamide (also known as nylon, such as nylon 6 and nylon 6.6), polyester (e.g., polyethylene terephthalate), polytetrafluoroethylene (e.g., expanded polytetrafluoroethylene), polyether-ester such as polybutester (block copolymer of butylene terephthalate and polytetra methylene ether glycol), polyurethane, polypropylene, polyethelene, and the like.

Sutures made of non-degradable suture material are particularly suitable for applications in which the suture is meant to remain permanently or is meant to be physically removed from the body. In certain embodiments, the non-degradable suture material may comprise or include any other polymer useful for suturing applications that currently exists or that may be developed in the future.

The suture material or thread may be mono-filamentary, i.e., formed of a single filament, or multi-filamentary, i.e., formed from a combination of two or more filaments, e.g., three filaments arranged in a braided fashion. ., formed from a combination of two or more filaments, e.g., three filaments arranged in a braided/twisted fashion within a smooth outer sleeve of extruded material. The suture thread has a length, where that length is typically at least 5 inches, or at least 10 inches, or at least 15 inches, or at least 20 inches. The suture thread will typically have two ends, which may be described as a deployment end and/or a trailing end. In such a case, the deployment end of the suture thread is that end that first enters tissue, adjacent to the needle, such as connected via the thread attachment component to the distal end of the needle body of the needle. Alternatively, the suture material may be looped, such that each of the two free ends are connected to the needle by the thread attachment component.

The thread can be a suture, which can be non-absorbable or absorbable of various gauges. The thread can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, or other natural or artificial materials. The suture thread may comprise metallic wire suture (as used for sternal closure during open heart surgery) or any other biocompatible material used for approximating human or animal tissues. The thread can also comprise any material used to stitch or otherwise approximate, or otherwise connect in any way, inanimate objects of any sort. The thread is solid, but can be perforated. The thread is internally dense, but can be hollow. The thread can be rigid, semi-rigid, elastic, resilient, or flexible. For example, the thread can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The thread can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The suture material or thread may be bioabsorbable, such that after introduction into a tissue it is broken down and absorbed by the body. Typically, the degradation process is at least partially mediated by, or performed in, a biological system. Accordingly, bioabsorbable refers to a chain scission process by which a polymer chain is cleaved through various mechanisms, including, for example, by chemical reaction (e.g., hydrolysis, oxidation/reduction, enzymatic mechanisms or a combination of these) or by a thermal or photolytic process. Bioabsorbable suture material may include polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™[glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). In certain embodiments, the bioabsorbable suture material may comprise or include any other polymer useful for suturing applications that currently exists or that may be developed in the future.

Alternatively, the suture material or thread may be non-degradable, such that it is not degraded by chemical, thermal, or photolytic process. Non-degradable suture material includes polyamide (also known as nylon, such as nylon 6 and nylon 6.6), polyester (e.g., polyethylene terephthalate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Sutures made of non-degradable suture material are particularly suitable for applications in which the suture is meant to remain permanently or is meant to be physically removed from the body. In certain embodiments, the non-degradable suture material may comprise or include any other polymer useful for suturing applications that currently exists or that may be developed in the future.

The suture material or thread may comprise a coating or agent applied to a surface thereof that may affect wound healing, such as a coating material, wound healing agent, antimicrobial agent, antibacterial agent, growth factor, adhesive, sealant, blood product, blood component, preservative, anti-adhesive, protein, polysaccharide, peptide, genetic material, viral vector, nucleic acid, nucleotide, plasmid, lymphokine, radioactive agent, metal, alloy, salt, growth factor, growth factor antagonist, cell, hydrophobic agent, hydrophilic agent, immunological agent, anti-colonization agent, and combinations thereof. The suture material or thread may comprise a coating or agent applied to a surface thereof that may enhance the surgeon's ability to accurately suture, such as colorants, dyes, ultraviolet absorbers, ultraviolet stabilizers, photochromic agents, diagnostic agent, imaging agent, radiopaque agent, or combinations thereof. The suture material or thread may comprise a coating or agent applied to a surface thereof that may reduce the friction or drag of the suture material or thread as it is passes through tissue.

The suture material or thread may have a diameter indicated by a cross-sectional dimension. This diameter may be determined at a location along the suture where there are either no barbs, or the barbs that are present are pushed against the suture body so that they are flush with the surface of the suture body. The suture may have no barbs or may have barbs along all or only a portion of the suture length. According to certain embodiments, the suture may have at least one barbed region positioned distal from the thread attachment component. Such positioning may allow the presently disclosed suture needle to be backed out of a suture site, wherein the suture material adjacent the needle would not have barbs and would easily slide back through the tissue. In some embodiments the tapered portion covers the barbs on the suture, thereby allowing the suture needle to be backed out of the suture sight. More distally located barbed suture may remain securely in place once positioned in the tissue.

The suture may have a generally circular cross-sectional shape, or may have a non-circular shape, e.g., polygonal such as 3-sided (triangular), or 4-, 5- or 6-sided (hexagonal) sided or a shape described by a polygon with any number of sides. The cross section of the suture body may have an oval, an ellipsoid, an oblong, or a semi-circular appearance.

Suture sizing is based upon diameter. The United States Pharmacopeia (“USP”) designation of suture size runs from 0 to 7 in the larger range and 1-0 to 11-0 in the smaller range; in the smaller range, the higher the value preceding the hyphenated zero, the smaller the suture diameter. Under the USP nomenclature system, the actual diameter of a suture will depend on the suture material, so that, by way of example, a suture of size 5-0 and made of collagen will have a diameter of 0.15 mm, while sutures having the same USP size designation but made of a synthetic absorbable material or a non-absorbable material will each have a diameter of 0.1 mm. The selection of suture size for a particular purpose depends upon factors such as the nature of the tissue to be sutured and the importance of cosmetic concerns; while smaller sutures may be more easily manipulated through tight surgical sites and are associated with less scarring, the tensile strength of a suture manufactured from a given material tends to decrease with decreasing size. It is to be understood that the suture materials for use with the suture needles disclosed herein include without limitation 7, 6, 5, 4, 3, 2, 1, 0, 1-0, 2-0, 3-0, 4-0, 5-0, 6-0, 7-0, 8-0, 9-0, 10-0 and 11-0. It is to be understood that a variety of suture lengths may be used with the suture needles described herein.

The needle body may have any profile known in the art, such as circular, oval, triangular, and the like. The needle body may have a smooth surface. According to certain embodiments, the needle body may be a non-smooth profile. For example, when trying to drive needle through a tissue using a rotational needle drive, a smooth surface of the needle body may allow the needle to deviate out of an initial chosen plane of rotation, especially for needle bodies having an oval shape. Moreover, it is not possible to securely position these needles in a non-standard angle. Accordingly, an object of the present disclosure is to provide suture needles that may perform optimally in roller needle drivers and may incorporate features that enable multi-planar needle driving.

Rotational needle drivers generally comprise an actuator portion having linear and rotational motion systems and an interactive portion configured to interact with the suture needle. Each interactive portion comprises a first extended member and a second extended member which approximate a pair of rod-like scissors that may be opened and closed. Each extended member comprises a main needle-grasping portion having grooves. The suture needles of the present disclosure are designed to include ridges that mate with these grooves of the rollers so that a secure connection between the needle and the rollers may be achieved.

When the suturing procedure begins, the suture needle having a non-smooth surface configured to interact with the grooves of the two rollers is grasped at a desired angle. The surgeon may press an actuator on the needle driver that brings the rollers closer so that they may grasp the needle and may lock the rollers in position exerting the necessary force to maintain the needle still. Once the needle is tightened, the actuator may create a rotational movement of the rollers, wherein the rollers rotate in directions opposite from each other to affect movement of the needle in either of two directions. Thus, the surgeon may drive the needle through the tissue as the rollers rotate. When the desired rotation is completed, the surgeon releases the locking mechanism. This method is repeated on the other tissue that is to be joined to the tissue already perforated by the needle, and each step above is repeated as many times as stitches have to be performed.

The design of the roller needle driver permits rotation of a curved suture needle without requiring a rotational motion of the wrist. Therefore, the instrument can be held in a position that may not allow wrist rotation and still carry out its function. In some situations, the structures to be sutured lie very deep. Doing surgery on the vertebral column of very obese patients is one such situation. For example, if the dura mater (a membrane that protects and envelops the spinal cord) is cut it needs to be sutured. The dura mater lies within a deep constricted space. In a very obese patient, the additional thickness of the adipose (fatty) tissue makes the dura lie even deeper from the surface. The surgeon may have to lean towards the patient and rotate the arm bearing the needle driver. This is necessary to get his forearm in a vertical position so he can maneuver the instrument properly. The needle driver used with a suture needle as disclosed herein may be operated in such a situation.

In certain embodiments, the suture needle includes ridges that extend along only a portion of the needle trunk. In certain embodiments, the suture needle includes ridges that begin at a first portion and end at a second portion of the needle trunk. In certain embodiments, the suture needle includes multiple discontinuous areas having ridges with intervening non-ridged areas. In certain embodiments, the suture needle includes certain areas with at least one of ridges, a groove surface, a roughened surface, a smooth surface, a bumpy surface, a ribbed surface, a tacky surface, or a polished surface. In certain embodiments, the suture needle includes certain areas with at least one of a ridged surface, a grooved surface, a roughened surface, a smooth surface, a bumpy surface, a ribbed surface, a tacky surface, or a polished surface, and other areas with at least one different surface. In certain embodiments, the ridged and non-ridged sections, or any combinations thereof may each be of any particular length, width, order, or orientation. In certain embodiments, the ridged surface, the grooved surface, the roughened surface, the smooth surface, the bumpy surface, the ribbed surface, the tacky surface, or the polished surface sections, or any combinations thereof may each be of any particular length, width, order, or orientation. In certain embodiments, there may be more than one type of non-ridged surface, such as, for example, a ridged surface, a grooved surface, a roughened surface, a smooth surface, a bumpy surface, a ribbed surface, a tacky surface, or a polished surface. For example, ridged sections, smooth sections and otherwise textured non-ridged sections can be combined in the same needle trunk for any type of application.

In certain embodiments, a suture needle may comprise a smooth tip section, ridged body and textured-non-ridged portion near the swage end that could induce some slight vibration captured by a robotic needle driver to improve haptic feedback to the surgeon on when to pull the needle out of the tissue to minimize tissue trauma, particularly in regions where tissue visibility or maneuverability is quite limited or tissues are very prone to tearing if the needle is pulled from the tissues too soon.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/−10% variation from a nominal value/term. Such variation is always included in any given value/term provided herein, whether or not such variation is specifically referred thereto.

In particular, the presently disclosed suture needles can be used for an open surgery, a minimally invasive surgery, a laparoscopic surgery, or an end effector robotic surgery. As such, the presently disclosed suture needles can be used for manual surgery or automated surgery. Some examples of surgeries where the presently disclosed suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, fetal surgery or bariatric surgery, or among many others in human (e.g., between newborn until 120 years old, male, female) or animal (e.g., mammal, birds, fish, land animals) applications. However, note that the presently disclosed suture needles can also be applied to non-medical applications, such as garment making, fabric stitching, knot applications, sowing, shoe making, or others.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Similarly, as used herein, various singular forms “a,” “an” and “the” are intended to include various plural forms as well, unless context clearly indicates otherwise. For example, a term “a” or “an” shall mean “one or more,” even though a phrase “one or more” is also used herein.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed below could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context or the prior art.

Although preferred embodiments have been depicted and described in detail herein, skilled artisans know that various modifications, additions, substitutions and the like can be made without departing from the spirit of this disclosure. As such, these are considered to be within the scope of the disclosure, as defined in the following claims.

1. A suturing needle comprising:
a needle tip portion having a needle tip at a distal end for piercing tissue;
a needle body having a distal end connected to a proximal end of the needle tip portion;
a hollow thread attachment component having a distal end attached to a proximal end of the needle body, the thread attachment component comprising a viewing port,
a hollow extension ramp portion having a distal end and a tapered portion at a proximal end, the distal end attached to a proximal end of the thread attachment component;
a hollow flexible extension component having a distal end and a proximal end, the distal end connected to the proximal end of the thread attachment component, and the proximal end attached to the distal end of the tapered extension ramp portion; and
a suture thread attached to the thread attachment component.

2. The suturing needle of claim 1, wherein the distal end of the ramp has a diameter equal to the needle body diameter, and the proximal end of the ramp has a diameter equal to a suture thread diameter, and wherein the tapered portion provides a smooth transition from the proximal end of the ramp portion to the distal end thereof.

3. The suturing needle of claim 1, wherein the hollow flexible extension component has a diameter equal to the needle body diameter.

4. The suturing needle of claim 1, wherein the hollow flexible extension component has a diameter smaller than a needle body diameter.

5. The suturing needle of claim 1, wherein the hollow flexible extension component comprises a plurality of projections and indentations arranged in an alternating pattern.

6. The suturing needle of claim 5, wherein each of the plurality of indentions comprises a polymer.

7. The suturing needle of claim 1, wherein the hollow flexible extension component comprises a plurality of tubes arranged longitudinally along an outer circumference of the hollow flexible extension component.

8. The suturing needle of claim 7, wherein each of the plurality of tubes includes a first end having a convex shape and a second end opposite to the first having a concave shape.

9. The suturing needle of claim 8, wherein the plurality of tubes are arranged such that the first end of a tube is positioned adjacent to a second end of a tube to form an interlocking arrangement.

10. The suturing needle of claim 1, wherein the hollow flexible extension component is surrounded by a braided metal sheath arranged longitudinally along an outer circumference of the hollow flexible extension component.

11. The suturing needle of claim 1, wherein an outer circumference of the flexible extension component includes longitudinally offset indentations.

12. The suturing needle of claim 1, wherein a plurality of polymer beads are arranged along an outer circumference of the hollow flexible extension component.

13. The suturing needle of claim 12, wherein the plurality of beads are arranged such that adjacent beads are in contact with each other.

14. The suturing needle of claim 12, wherein each of the plurality of beads has a shape independent selected from an oval, an ellipsoid, an oblong, a circular or a semi-circular shape.

15. The suturing needle of claim 1, wherein a plurality of tubes and beads are arranged longitudinally along a circumference of the flexible extension component.

16. The suturing needle of claim 15, wherein the plurality of tubes and beads are arranged in an alternating pattern.

17. The suturing needle of claim 15, wherein a bead is arranged between two adjacent tubes.

18. The suturing needle of claim 1, wherein the hollow flexible extension component comprises metal or metal alloy microbeads in an inner hollow portion.

19. The suturing needle of claim 18, wherein the microbeads are arranged such that the suture thread passes through the flexible extension components.

20. The suturing needle of claim 18, wherein the microbeads are arranged in a single layer.

21. The suturing needle of claim 18, wherein the microbeads are arranged in multiple layers.

22. The suturing needle of claim 1, wherein an outer circumference of the flexible extension components is surrounded by protective layer.

23. The suturing needle of claim 22, wherein the protective layer comprises an injection-molded polymer.

24. The suturing needle of claim 1, wherein the hollow flexible extension component comprises a chain link structure.

25. The suturing needle of claim 24, wherein the tapered extension ramp portion is formed continuously with the hollow flexible extension component.

26. The suturing needle of claim 1, wherein the hollow flexible extension component comprises a metal or metal alloy tube lined on an inner surface thereof with a polymer filling.

27. The suturing needle of claim 1, wherein the hollow flexible extension component comprises a nickel-titanium wire.

28. The suturing needle of claim 1, wherein the suture thread is attached to the thread attachment component by swaging when the suture thread is visible through the viewing port.

29. The suturing needle of claim 1, wherein the suture thread is mono-filamentary or multi-filamentary.

30. The suturing needle of claim 1, wherein the suture thread is bioabsorbable.

31. The suturing needle of claim 1, wherein the suture thread comprises at least one region of tissue retaining structures.

32. The suturing needle of claim 31, wherein the tissue retaining structures are barbs.

33. The suturing needle of claim 31, wherein the at least one region of tissue retaining structures are distal from the tapered portion of the ramp portion.

34. The suturing needle of claim 1, wherein the needle tip portion is formed to become gradually thicker from the needle tip to the proximal end thereof.

35. The suturing needle of claim 1, wherein the needle body is straight.

36. The suturing needle of claim 1, wherein the needle body curved.

37. The suturing needle of claim 1, wherein the needle body is formed to have a substantially circular cross-section.

38. A method of suturing tissues using a roller needle driver, the method comprising the steps of:
(a) providing a suture needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a ramp portion having a distal end and a tapered portion at proximal end, the distal end attached to a proximal end of the thread attachment component; a hollow flexible extension component having a distal end and a proximal end, the distal end connected to the proximal end of the thread attachment component, and the proximal end attached to the distal end of the tapered extension ramp portion; and a suture thread attached to the thread attachment component;
(b) grasping, between rollers of the roller needle driver, the suture thread or the tapered portion of the ramp portion distal from the needle body;
(c) loading the needle so that the rollers are positioned on the needle body without opening the rollers;
(d) driving the needle through a tissue being subjected to suture;
(e) moving the needle through the tissue without releasing the needle from the tissue;
(f) grasping the suture needle at an end exiting from the tissue through which the needle was moved; and
(g) repeating steps (d)-(f) until completion of the suturing process.

39. A suturing needle comprising:
a needle tip portion having a needle tip at a distal end for piercing tissue;
a needle body comprising a curved portion having a distal end connected to a proximal end of the needle tip portion and proximal end, wherein the curved portion comprises at least one of a flexible section or a non-flexible section, and a straight portion having a distal end coupled to the proximal end of the curved portion and a proximal end, wherein the straight portion comprises at least one flexible section and at least one non-flexible section;
a hollow thread attachment component having a distal end attached to at least one of the proximal end of the straight portion or a portion of the needle body between the distal end of the curved portion and the proximal end of the straight portion, and between a flexible section and a non-flexible section; and
a suture thread attached to the thread attachment component.

40. The device of claim 39, wherein the straight portion further comprising, a hollow extension component having a distal end and a proximal end, the distal end connected to the proximal end of curved portion, and the proximal end attached to the hollow thread attachment component.
